(19) United States
(12) Patent Application Publication
(10) Pub. No.: US 2003/0032020 A1
(43) Pub. Date: Feb. 13, 2003

(54) Polymorphic DNA fragments and uses thereof

(76) Inventor: Sydney Brenner, La Jolla, CA (US)

Correspondence Address: PERKINS COIE LLP, P.O. BOX 2168, MENLO PARK, CA, 94026, US

(21) Appl. No.: 09/934,020

(22) Filed: Aug. 21, 2001

Related U.S. Application Data
(60) Provisional application No. 60/227,058, filed on Aug. 21, 2000.

(51) Int. Cl. C12Q 1/68; C12P 19/34

(52) U.S. Cl. 435/6; 435/91.2

(57) ABSTRACT

The invention provides methods and materials for generating a reference library of restriction fragments from pooled nucleic acids that contain a sequence polymorphism. An important aspect of the invention is the use of the reference population of restriction fragments to compare the frequencies of polymorphic sequences between different population pools. Such comparisons may be accomplished by competitively hybridizing DNA from the respective pools which has been enriched for the presence of a restriction site polymorphism with DNA from the reference population. Preferably, such competitive hybridization reactions are carried out on the reference library attached to one or more solid phase supports. Most preferably, members of the reference library are attached to individual microparticles so that each microparticle has a unique fragment attached. After competitive hybridization, the microparticles may be analyzed and sorted to identify those microparticles carrying sequences for which the pools being compared exhibit different polymorphic frequencies.

This application claims the benefit of U.S. Provisional Application No. 60/227,058 filed Aug. 21, 2000, which is incorporated herein by reference in its entirety.

### FIELD OF THE INVENTION

The invention relates generally to methods for isolating polymorphic DNA fragments from genomes or other nucleic acid populations, and more particularly, to a high-throughput method of isolating restriction fragments containing polymorphic sequences and using such fragments for genetic identification and comparison.

### BACKGROUND OF THE INVENTION

Genetic factors contribute to virtually every disease, conferring susceptibility, resistance, or influencing interaction with environmental factors, Collins et al. (1997), Science 278:1580-1581. As genome mapping and sequencing projects advance, more attention is being directed to the problem of determining sequence variability between genomes of different individuals. In the area of human health, it is believed that a detailed understanding of the correlation between genotype and disease susceptibility, responsiveness to therapy, likelihood of side-effects, and other complex traits, will lead to improved therapies, improved application of existing therapies, better preventative measures, and better diagnostic procedures, Caskey (1987), Science 236:1223-1229; White and Caskey (1988), Science, 240:1483-1488; Lander et al. (1994), Science, 265:2037-2048; Schafer et al. (1998), Nature Biotechnology, 16:33-39; and Housman et al. (1998), Nature Biotechnology, 16:492-493.

Many techniques are available for detecting the presence or absence of suspected mutations or polymorphic sequences, including direct sequencing, ligation-based assays, restriction fragment length analysis, multiplexed and/or allele-specific polymerase chain reaction, assays based on differential electrophoretic mobilities, primer extension, mismatch repair enzymes, and specific hybridization, e.g., Taylor, Editor, Laboratory Methods for the Detection of Mutations and Polymorphisms in DNA (CRC Press, Boca Raton, 1997); Cotton, Mutation Detection (Oxford University Press, Oxford, 1997); Landegren et al. (1988), Science, 242:229-237; Landegren et al. (1998), Genome Research, 8:769-776 (1998); Brown (1994), Current Opinion in Genetics and Development, 4:366-373 (1994); Shumaker et al. (1996), Human Mutation, 7:346-354; Nikiforov et al. (1994), Nucleic Acids Research 22:4167-4175; Pastinen et al. (1997), Genome Research 7:606-614; Shuber et al. (1997), Human Molecular Genetics 6:337-347; and the like. However, most of these techniques are not directed to large-scale identification, or surveying, of polymorphic sequences throughout whole genomes, and several of the above techniques require that the polymorphisms be known beforehand. This limitation is significant, as the frequency of single nucleotide polymorphisms in unrelated individuals has been estimated to average as high as once every several hundred basepairs, e.g., Cooper et al. (1985), Human Genetics 69:201-205; Wang et al. (1998), Science 280:1077-1082. Thus, the number of possible sequence differences between individuals is enormous, and the task of finding significant differences, e.g., those associated with disease conditions, would be extremely difficult using techniques that are applicable to only one or a few polymorphic sequences at a time.

Although several techniques have been developed for large-scale comparisons of genomes, including representational difference analysis (RDA), e.g., Lisitsyn et al. (1993), Science 259:946-951, genome mismatch scanning (GMS), e.g. Nelson et al. (1993), Nature Genetics 4:11-18, and microarray-based methods, e.g., Wang et al. (Id.), and Winzeler et al. (1998), Science 281:1194-1197, each of these techniques has significant limitations. RDA entails repeated cycles of hybridizing highly complex mixtures of DNA and amplifying the products of such hybridizations with polymerase chain reaction (PCR). As the name of the technique indicates, the DNA involved in these operations is only a small portion of the genomes being compared (about 10%, Aldhous (1994), Science, 265:2008-2010) because of the difficulty of amplifying large fragments with PCR. Also, because of the complexity and size of the fragments in the hybridization reactions, it is not clear how effective the technique is in isolating subtle, yet pervasive, differences such as single nucleotide polymorphisms complements (GMS also requires the hybridization of highly complex mixtures of DNA fragments, but more importantly the objective of the technique is to identify identical sequences in two populations; thus, it has limited applicability in analyses requiring the identification of differences, such as genetic association studies. GMS further requires the use of mismatch recognition enzymes which may have widely varying sensitivities depending on the type of enzyme employed and the type of mismatch present, e.g., Cotton (Id.). Finally, both GMS and microarray-based methods employ arrays of DNAs complementary to the processed sequences as their primary measurement tool. Thus, the sequences suspected of being the same, in the case of GMS, or of containing polymorphisms, in the case of direct detection by micro arrays, must be known before hand.

In view of the above, it would be highly desirable if there was an approach available that permitted rapid and sensitive genome-wide identification of differences in genetic composition between groups of individuals.

### SUMMARY OF THE INVENTION

In accordance with the objects outlined above, the present invention provides compositions and methods for forming nucleic acid reference libraries from pooled genomic DNA. The reference libraries are heterogeneous mixtures enriched for polymorphic nucleic acid fragments. The polymorphic nucleic acid fragments hybridize to subregions of the pooled DNA which have a restriction site polymorphism.

The methods for making the reference libraries comprise the steps of (1) digesting pooled genomic DNA with a first restriction endonuclease to form first restriction fragments; (2) forming a first population of single stranded restriction fragments from the first restriction fragments which contain a restriction site for a second restriction endonuclease; (3) forming a second population of single stranded restriction fragments from the first restriction fragments which lack restriction sites for the second restriction endonuclease; (4) hybridizing the first and second populations of single stranded DNA fragments to form a population of duplexes; and (5) isolating the duplexes to form a reference library. The library which is obtained is enriched for fragments which hybridize to genomic subregions which are polymorphic as to the restriction site for the second restriction enzyme.

The invention further provides methods for determining the ratio of such polymorphic subregions as between different populations. The methods provide a significant improvement over conventional marker associated studies, as no sequence information is required to generate and use the reference libraries. Briefly, pooled DNA from first and second pooled test populations is digested with a first restriction endonuclease. The populations are then enriched for those fragments having a polymorphism associated with the restriction site for a second restriction endonuclease. The enriched populations are then contacted with a reference library which is preferably created as described above using the same restriction endonucleases. Differences in the extent of hybridization provide an indication of the ratio or frequency of the different polymorphisms as between the two pools of DNA. In some embodiments, such differences can be correlated with observed differences in phenotype between the two populations.

### BRIEF DESCRIPTION OF THE DRAWINGS

FIGS. 1A-D illustrates the concept of a reference library.

FIGS. 2A-D illustrates a preferred scheme for generating a reference population of polymorphic fragments.

FIG. 3 schematically illustrates a method for generating labeled probes from each of two pools of genomic DNA for competitively hybridizing to a reference population of restriction fragments.

FIG. 4 schematically illustrates a method for attaching populations of identical tag-fragment conjugates to microparticles.

FIGS. 5A and B illustrate a preferred method for attaching fragments of a reference population to microparticles.

FIGS. 6A and B illustrate a preferred method for isolating fragments for sequencing after sorting by fluorescence-activated cell sorter (“FACS”).

FIG. 7A shows restriction site maps of the two pUC19 plasmids of Example 1.

FIG. 7B is an electropherogram showing the isolation of fragments of the expected sizes formed from the Sau 3A restriction fragment containing the Taq I polymorphism.

FIG. 8A illustrates the reaction scheme for producing single stranded Taq⁺ fragments from Sau 3A digested pUC19 plasmids.

FIG. 8B illustrates the reaction scheme for producing single stranded Taq⁻ fragments from Sau 3A digested pUC19 plasmids.

FIG. 8C illustrates the reaction scheme for recovering double stranded Sau 3A fragments that are polymorphic with respect to Taq I.

FIGS. 9A and B illustrate the reaction scheme for producing single stranded Tai⁺ fragments from Bst YI digested human DNA.

FIGS. 10A and B illustrate the reaction scheme for producing single stranded Tai⁻ fragments from Bst YI digested human DNA.

FIG. 11 illustrates the reaction scheme for producing a reference SNP library from Tai⁺ and Tai⁻ fragments.

FIG. 12 illustrates the scheme for generating a reference population of polymorphic fragments using single strand endonuclease.

### DETAILED DESCRIPTION OF THE INVENTION

The invention is directed to reference libraries of nucleic acid fragments which are associated with nucleic acid polymorphisms. Such libraries are useful in identifying single or multiple alleles which are associated with different phenotypes. In practice the reference library is generated based upon polymorphisms within a restriction site for a restriction endonuclease.

Briefly, such a library is preferably formed by digesting genomic DNA from a pool of individuals with a first and a second restriction endonuclease to form a population of restriction fragments; isolating restriction fragments of the population digested by both the first and second restriction endonucleases and forming a first single stranded fragment population therefrom; separately isolating restriction fragments from the population digested by the first restriction endonuclease but not the second restriction endonuclease and forming a second single stranded fragment population therefrom; hybridizing the first and second single stranded fragment populations to form a population of duplexes; and isolating the population of duplexes to form a reference library of restriction fragments that contain sequence polymorphism.

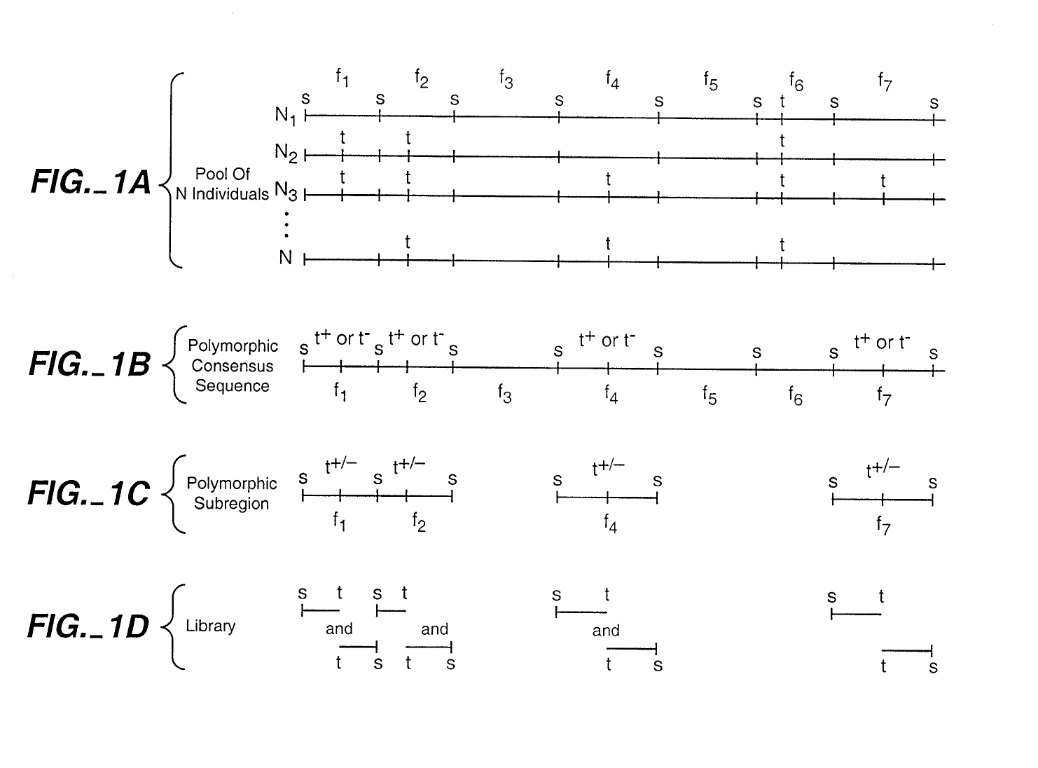

The reference library which is made up of a mixture of heterogeneous nucleic acid fragments can be described by reference to FIG. 1. FIG. 1 depicts the relationship of various components of the invention as they relate to restriction endonuclease polymorphisms associated with one or more restriction enzymes. In FIG. 1A, theoretical genomic DNA from a pool of N individuals are aligned to provide maximum homology among the sequences. Genomic DNA from four individuals is shown in FIG. 1. In FIG. 1A, first endonuclease restriction sites s are shown which can be recognized and/or cleaved by enzyme S. In addition, second endonuclease restriction cleavage sites t are shown which are capable of being recognized and/or cleaved by restriction endonuclease T. The regions spanning first restriction sites s correspond to subregions f₁ through f₇. When genomic DNA from each of the individuals is combined as a mixture and digested with restriction endonuclease S, a population of restriction fragments corresponding to the subregions f₁ through f₇ is formed.

As among the sequences shown in FIG. 1A, some subregions contain no t restriction endonuclease sites (e.g., f₃ and f₅), whereas other subregions contain the t restriction endonuclease site in all instances, e.g., f₆. Other subregions contain differences amongst the individuals as to whether or not the t restriction site is present. See, e.g., f₁, f₂, f₄ and f₇. If each of these restriction sites are projected onto a single theoretical sequence, the polymorphic consensus sequence of FIG. 1B is obtained. Subregions f₁ through f₇ are shown for comparative purposes. In the case of subregions f₁, f₂, f₄ and f₇, restriction site t is shown as being either present or absent, i.e., t^{+/-}. Subregions f₁, f₂, f₄ and f₇ are shown in FIG. 1C vis-a-vis their relationship to the polymorphic consensus sequence and the sequence as set forth in FIG. 1A. These subregions, sometimes referred to as “polymorphic subregions”, define the reference library.

The reference library is shown in FIG. 1D. As can be seen the library comprises fragments which comprise portions of the polymorphic subregions. As explained in more detail hereinafter, the method for generating this library results in enrichment for fragments other than those located between the polymorphic subregions. The library is thus skewed to have subregions f₁, f₂, f₄ and f₇ over-represented while subregions f₃, f₅ and f₆ are under-represented or absent. The net effect is to decrease the complexity of the library which would otherwise be obtained by a simple double digest of the pooled genomic library with S and T. This provides a library which can be used to test other populations for polymorphisms at the t restriction site which may be associated with different phenotypes.

The reference libraries are enriched for fragments other than those located between polymorphic subregions. By “enriched” herein is meant that relative to the polymorphic subregions some or all of the fragments corresponding to non-polymorphic subregions have been selected against in the methods of the invention. Referring to FIG. 1A, non-polymorphic subregions are those which contain no t restriction endonuclease site (e.g., f₃ and f₅), and those which contain the t restriction endonuclease site in all instances (e.g., f₆). As used herein, non-polymorphic fragments are not necessarily the same as non-polymorphic subregions.

In a preferred embodiment, 50 percent of the non-polymorphic subregions are removed. Preferably, 75 percent of the non-polymorphic subregions are removed. More preferably, 90 percent of the non-polymorphic subregions are removed, leaving a library substantially free of non-polymorphic subregions.

In a preferred embodiment, the reference library is made up of fragments of DNA corresponding to polymorphic subregions derived from a pool of individuals which is large enough so as to maximize the presence of the gene pool of a particular population. Preferably, the starting pool of nucleic acids contains 50 percent of the alleles in a given population; more preferably, 75 percent; more preferably, 90 percent; and most preferably, 95 percent.

The number of different individuals used as a source to form the nucleic acid pool from which the reference library is made determines the number of polymorphisms and alleles present in the library at given locus. For example, if a few individuals are used, only a limited number of polymorphisms may be present. Similarly, loci in linkage disequilibrium with such polymorphisms may be absent from the library. On the other hand, if many individuals are used, a greater representation of the polymorphisms present in the population will be found in the reference library. Preferably, the starting nucleic acid pool is obtained from the same species, such as humans, primates, bovine, ovine, porcine, etc. Similarly, nucleic acid can be pooled from various plant species as well as from various eukaryotic and prokaryotic organisms.

It is preferred that the reference library be made from a random population of nucleic acids so as to enhance the representation of polymorphisms in the library. However, in some embodiments, it may be desirable to use a nucleic acid pool containing nucleic acids selected from individuals having one or more defined phenotypes.

When used to analyze other populations, the polymorphic probes from the reference library are preferably used to compare the frequency of the various polymorphisms as between different pools of nucleic acids. By “polymorphic probe” herein is meant a nucleic acid fragment which comprises a portion of a polymorphic subregion. Such probes may comprise a fragment from the reference library or a sequence portion thereof. Portions of library fragments are preferably used if such sequences are unique. The reference library can be used in a number of ways. In one embodiment, DNA from one population may be pooled and compared to a second population. There is no need a priori for each population to be defined by a phenotype before using the reference library. However, in a preferred embodiment, each population is phenotypically defined so as to correlate differences in the observed polymorphism with differences in phenotype as between the two populations or as compared to the reference library. In some instances, the polymorphism may be in linkage disequilibrium with one or more alleles which permits the determination of haplotype associated with phenotype.

In a preferred embodiment using the reference library, a pool of DNA from individuals having a first phenotype is digested with first restriction endonuclease S to form a pool of restriction fragments. Fragments which are t⁻ are then selected. A second pool of DNA from individuals having an second phenotype is similarly treated to select for fragments which are also t⁻. The polymorphic probes are then contacted with the t⁻ enriched fragments and the relative frequency of the polymorphic subregions in the t⁻ population is determined. By way of example, referring to FIG. 1A, subregion f₁ is equally represented by the population of DNA from 4 individuals, half of the f₁ subregions are t⁺, the other half are t⁻. Assume that this is the first population. If, by way of example only, the second population contained only t⁻ f₁ subregions, the ratio of the signal obtained in the second t⁻ pool would be twice that obtained for the analogous pool from the first population. Such a difference would indicate that the t⁻ polymorphism has an association which may correlate with the observed difference in phenotype. Other associations may also be detected for one or more other polymorphic subregions.

An advantage of the present invention is that no sequence information is required to generate and use the reference libraries. All that is required is the use of at least two restriction enzymes which recognize and cleave different nucleic acid sequences. In the preferred embodiments, the restriction endonuclease cleavage results in “protruding ends” with at least 4 base-pair overhangs, as opposed to blunt ends, which can be used to further manipulate the restriction fragments as set forth in more detail in the methods which follow.

By “restriction site” is meant a region usually between 4 and 8 nucleotides within a nucleic acid, preferably a double stranded nucleic acid, comprising the recognition site and/or the cleavage site of a restriction endonuclease. Preferably, the recognition site and cleavage site are coextensive. A recognition site corresponds to a sequence within a nucleic acid which a restriction endonuclease or group of restriction endonucleases binds to. The cleavage site corresponds to the particular point of cleavage by the restriction endonuclease. In the case of double stranded nucleic acid it is preferable that the cleavage occur at a different position on the complementary strands so as to provide a protruding end. Depending on the restriction endonuclease, the cleavage site may be within the recognition site. However, some restriction endonucleases, e.g., type IIS have a cleavage site which is outside of the recognition site.

In a preferred embodiment, the polymorphisms which are used to generate the reference library are within a restriction site for a chosen enzyme. Thus, point mutations in the recognition and/or cleavage site can result in a restriction site which is no longer susceptible to cleavage by that particular endonuclease. Alternatively, the mutation can create a restriction site for an endonuclease. Polymorphisms such as insertions or deletions of one or more nucleotides can similarly result in resistance or susceptibility to digestion by a restriction endonuclease. Accordingly, the polymorphisms can correlate to the substitution, insertion or deletion of one or more nucleotides within a particular restriction site.

As used herein, the terms “mutation” and “polymorphism” are used somewhat interchangeably to mean a DNA molecule, such as a gene, that differs in nucleotide sequence from a reference DNA molecule, or wildtype, by one or more bases, insertions, and/or deletions. The usage of Cotton (supra) is followed in that a mutation is understood to be any base change whether pathological to an organism or not, whereas a polymorphism is usually understood to be a base change with no direct pathological consequences. In some instances, however, the polymorphism may be a mutation that produces a genotype associated with a particular phenotype.

Preferably, polymorphisms within a pool of nucleic acids are present at a given locus at the rate of at least 1%, e.g., for 1000 different nucleic acids in a pool, there are at least 10 nucleic acids containing the polymorphism at a given locus. More preferably, polymorphisms are present at a rate of 10% at a given locus. Each polymorphic locus therefore comprises a proper subset of the polymorphism, i.e., the subset contains at least one member of the locus with the polymorphism and at least one other member within the locus which lacks the polymorphism.

In a preferred embodiment, the reference library is made up of nucleic acid fragments. By “nucleic acid” herein is meant at least two nucleotides covalently linked together. A nucleic acid of the present invention will generally contain phosphodiester bonds, although in some cases, nucleic acid analogs are included that may have alternate backbones, comprising, for example, phosphoramide (Beaucage, et al. (1993), Tetrahedron 49(10):1925 and references therein; Letsinger (1970), J. Org. Chem. 35:3800; Sprinzl, et al. (1977), Eur. J. Biochem. 81:579; Letsinger, et al. (1986), Nucl. Acids Res. 14:3487; Sawai, et al. (1984), Chem. Lett. 805, Letsinger, et al. (1988), J. Am. Chem. Soc. 110:4470; and Pauwels, et al. (1986), Chemica Scripta 26:141), phosphorothioate (Mag, et al. (1991), Nucleic Acids Res. 19:1437; and U.S. Pat. No. 5,644,048), phosphorodithioate (Briu, et al. (1989), J. Am. Chem. Soc. 111:2321), O-methylphosphoroamidite linkages (see Eckstein, Oligonucleotides and Analogues: A Practical Approach, Oxford University Press), and peptide nucleic acid backbones and linkages (see Egholm (1992), J. Am. Chem. Soc. 114:1895; Meier, et al. (1992), Chem. Int. Ed. Engl. 31:1008; Nielsen (1993), Nature 365:566; Carlsson, et al. (1996), Nature 380:207, all of which are incorporated by reference). Other analog nucleic acids include those with positive backbones (Denpcy, et al. (1995), Proc. Natl. Acad. Sci. USA 92:6097), non-ionic backbones (U.S. Pat. Nos. 5,386,023; 5,637,684; 5,602,240; 5,216,141; and 4,469,863; Kiedrowshi, et al. (1991), Angew. Chem. IntL. Ed. English 30:423; Letsinger, et al. (1988), J. Am. Chem. Soc. 110:4470; Letsinger, et al. (1994), Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker, et al. (1994), Bioorganic & Medicinal Chem. Lett. 4:395; Jeffs, et al. (1994), J. Biomolecular NMR, 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins, et al. (1995), Chem. Soc. Rev., pp. 169-176). Several nucleic acid analogs are described in Rawls, C & E News, Jun. 2, 1997, page 35. All of these references are hereby expressly incorporated by reference. These modifications of the ribose-phosphate backbone may be done to facilitate the addition of additional moieties such as labels, or to increase the stability and half-life of such molecules in physiological environments. In addition, mixtures of naturally occurring nucleic acids and analogs can be made. Alternatively, mixtures of different nucleic acid analogs, and mixtures of naturally occurring nucleic acids and analogs may be made. A person skilled in the art will know how to select the appropriate analog to use in various embodiments of the present invention. For example, when digesting with restriction enzymes, natural nucleic acids are preferred.

Nucleic acids also may include nucleosides. By “nucleoside” herein is meant natural nucleosides, including 2′-deoxy and 2′-hydroxyl forms, e.g., as described in Kornberg and Baker, DNA Replication, 2nd Ed. (Freeman, San Francisco, 1992) and analogs. “Analogs” in reference to nucleosides include synthetic nucleosides having modified base moieties and/or modified sugar moieties, e.g., described by Scheit, Nucleotide Analogs (John Wiley, New York, 1980); Ulhlman and Peyman (1990), Chemical Reviews 90:543-584, or the like, with the only proviso that they are capable of specific hybridization. Such analogs include synthetic nucleosides designed to enhance binding properties, reduce complexity, increase specificity, and the like.

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc.

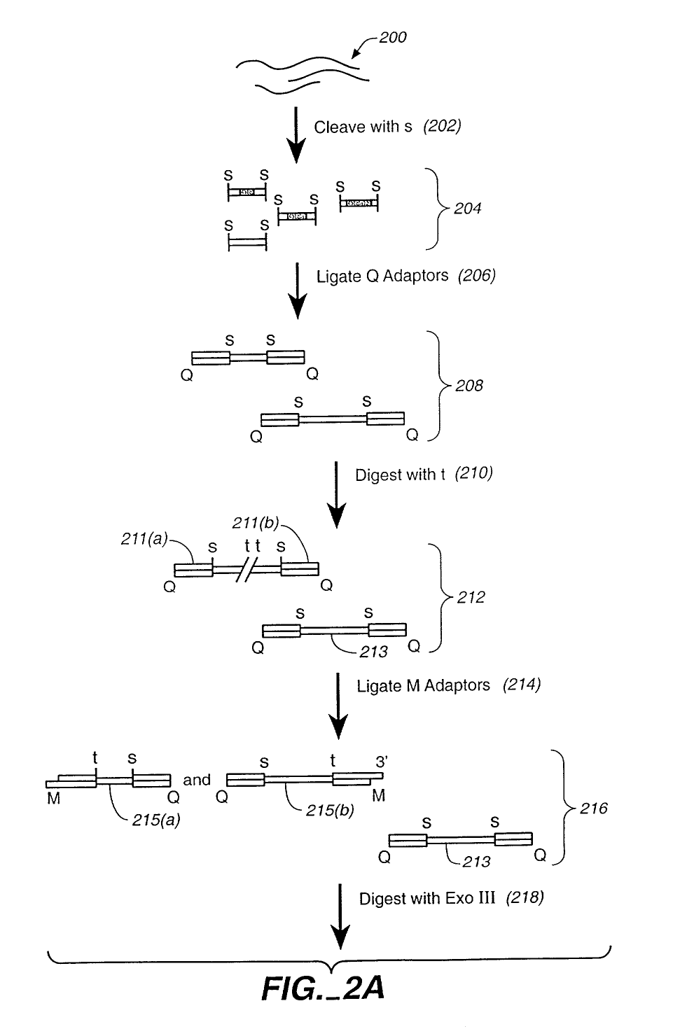

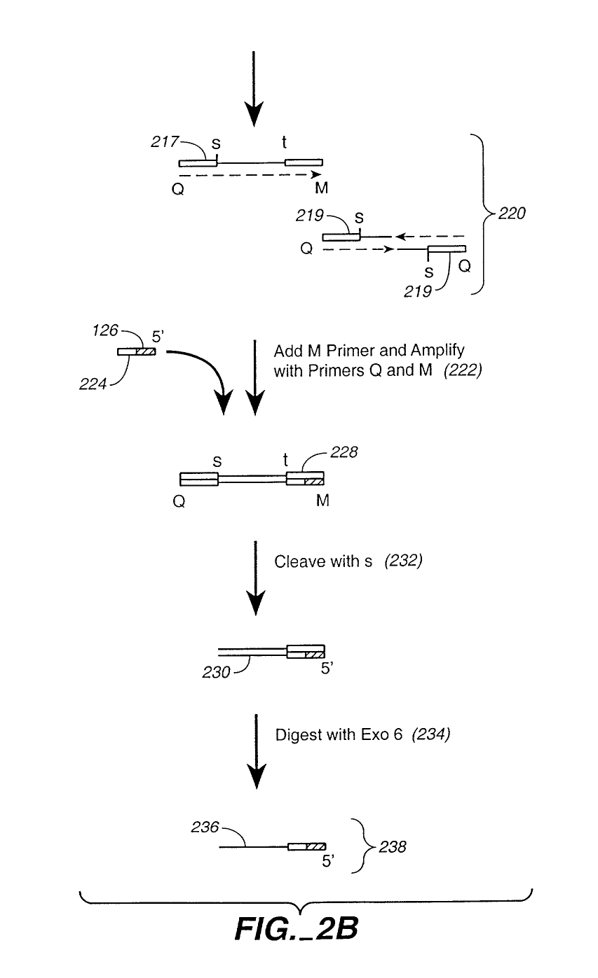

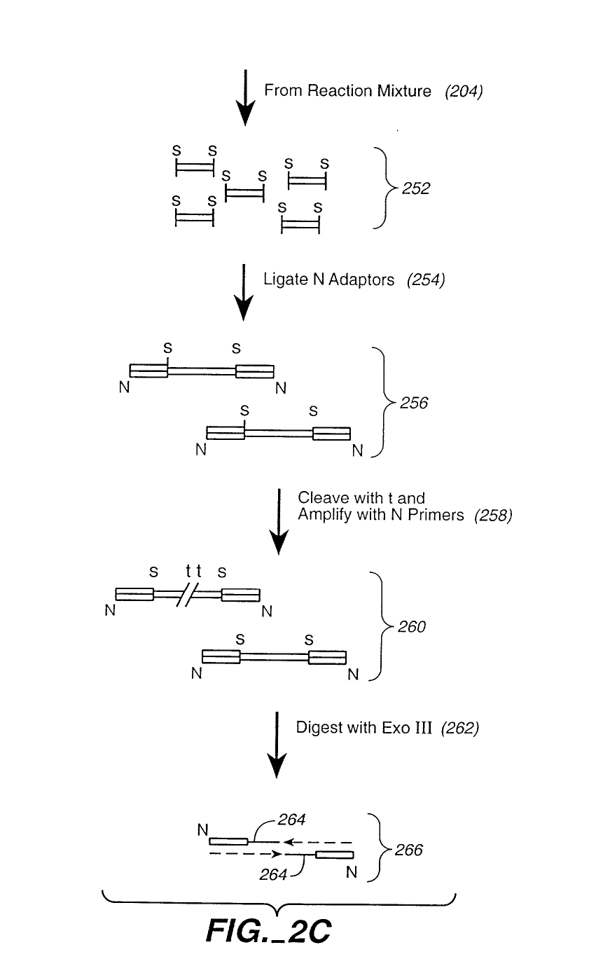

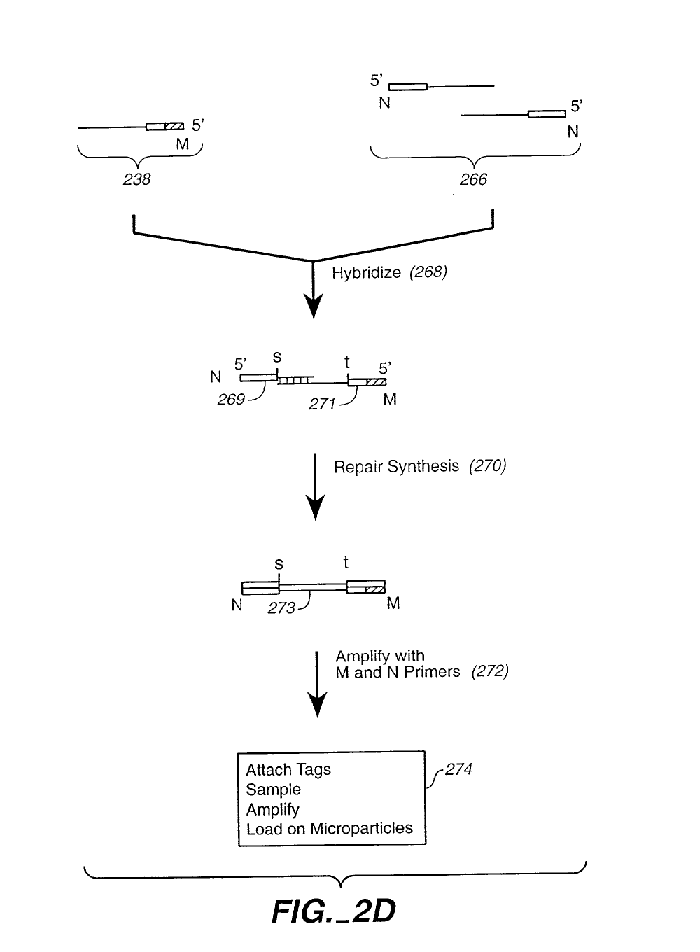

The following provides more detailed information with regard to the preparation of the reference libraries of the invention. In a preferred embodiment, a reference population of restriction fragments is produced by the method illustrated in FIGS. 2A through 2C. In FIG. 2A, genomic DNA (200) is extracted from each of the individuals of a population of interest and pooled. By “pooled nucleic acid” herein is meant combining nucleic acid such as the genomic DNA obtained from individuals in a population of interest such that a heterogeneous mixture of nucleic acid fragments is obtained when digested with at least two restriction endonucleases.

The number of individuals in the population is not critical; however, it is desirable to have the population sufficiently large so that many, if not all the polymorphic sequences of interest are captured. Preferably, the population consists of at least five individuals, and more preferably, it consists of at least ten individuals. Still more preferably, the population consists of a number of individuals in the range of from 10 to 100. When the genomic DNA is combined for processing, equal amounts are preferably contributed from each genome of the population. DNA (200) is cleaved (202) with a first restriction endonuclease S to produce a population of restriction fragments (204), to which Q adaptors are ligated (206) in a conventional ligation reaction to give fragment-adaptor complexes (208).

Restriction endonuclease S may be any restriction enzyme whose cleavage results in fragments with predictable protruding strands. Preferably, cleavage with first restriction enzyme S results in a protruding strand of at least four nucleotides. In further preference, restriction endonuclease S produces fragments having ends with 5′ protruding strands, which allows the 3′ recessed strands to be extended with a DNA polymerase in the presence of the appropriate nucleoside triphosphates. In a preferred embodiment, the 3′ recessed strands of such fragments are extended by one nucleotide to reduce the length of the protruding strands to three nucleotides, thereby destroying the self-complementarity of the protruding strand. This step helps to reduce self-ligation, both of the fragments and Q adaptors.

Q adaptors are conventional double stranded oligonucleotide adaptors which contain complementary protruding strands to those of restriction fragments (204). Q adaptors may vary widely in length and composition, but are preferably long enough to include a primer binding site for amplifying the fragment-adaptor complexes by polymerase chain reaction (PCR). Preferably, the double stranded region of Q adaptors is within the range of 14 to 30 basepairs, and more preferably, within the range of 16 to 24 basepairs.

Fragment-adaptor complexes (208) are digested (210) with second restriction endonuclease, T, to produce a population (212) of fragments (213) lacking a t restriction site and fragments (211) having a Q adaptor at one end and a protruding strand resulting from cleavage by T at the other end.

Restriction endonuclease T may be any restriction endonuclease different from S whose digestion of double stranded DNA leaves a protruding strand. Preferably, T is selected so that the frequency of t restriction sites in the target DNA is significantly less than that of the s restriction sites, thereby minimizing the probability that S-produced fragments have multiple internal t restriction sites. Preferably, most S-produced fragments have no more than one potential t restriction site. These conditions are satisfied by many combinations of restriction endonucleases, e.g., a restriction endonuclease that has a four basepair recognition site for S and a restriction endonuclease that has a six basepair recognition site for T.

For human DNA, preferably S is a restriction endonuclease which has a four nucleotide recognition site and whose cleavage results in a four nucleotide protruding strand, such as Sau 3A, Tsp 509 I, Nla III, or the like, and T is a restriction endonuclease which has a four nucleotide recognition site with a CG in its recognition sequence and whose cleavage results in at least a two nucleotide protruding strand, such as Taq I, Msp I, Hin P1, Hha I, Aci I, or the like. Because of the “CG” deficiency in human DNA, the frequencies of the latter enzyme recognition sites are much lower than that which would be expected in random sequence DNA. For example, the Taq recognition sequence occurs at frequency of about once every 1200 basepairs, rather than about once every 256 basepairs.

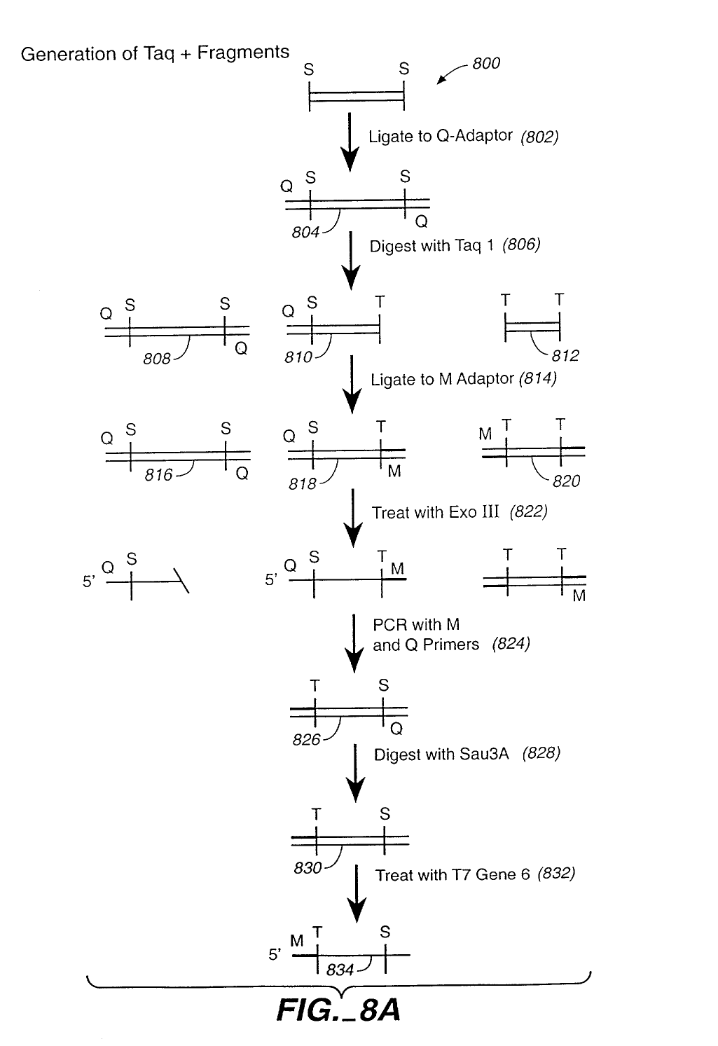

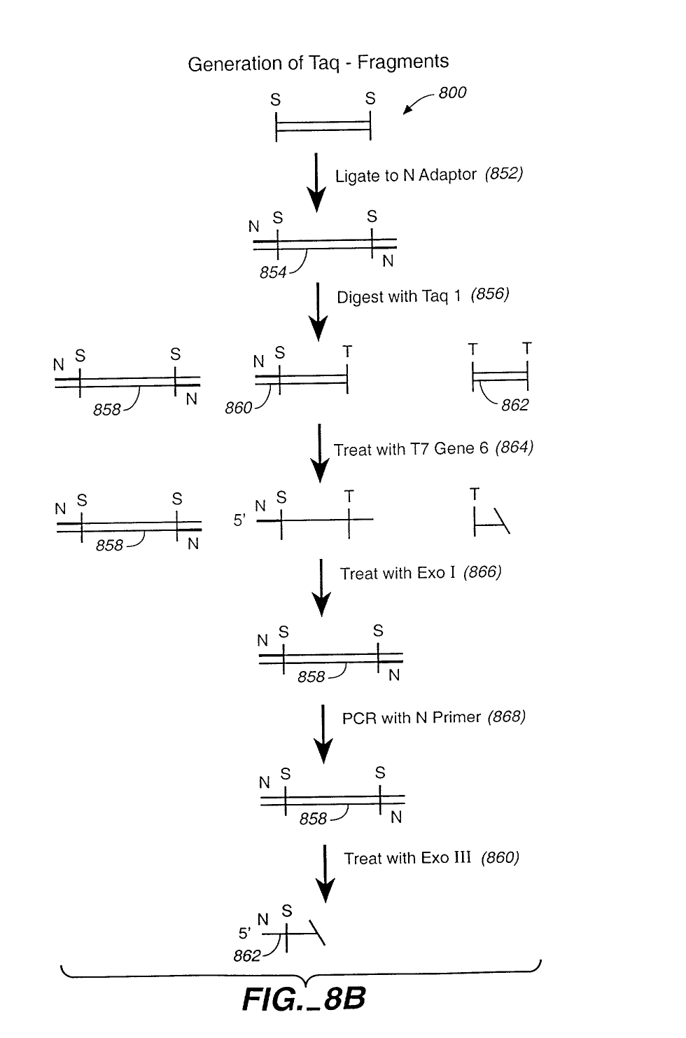

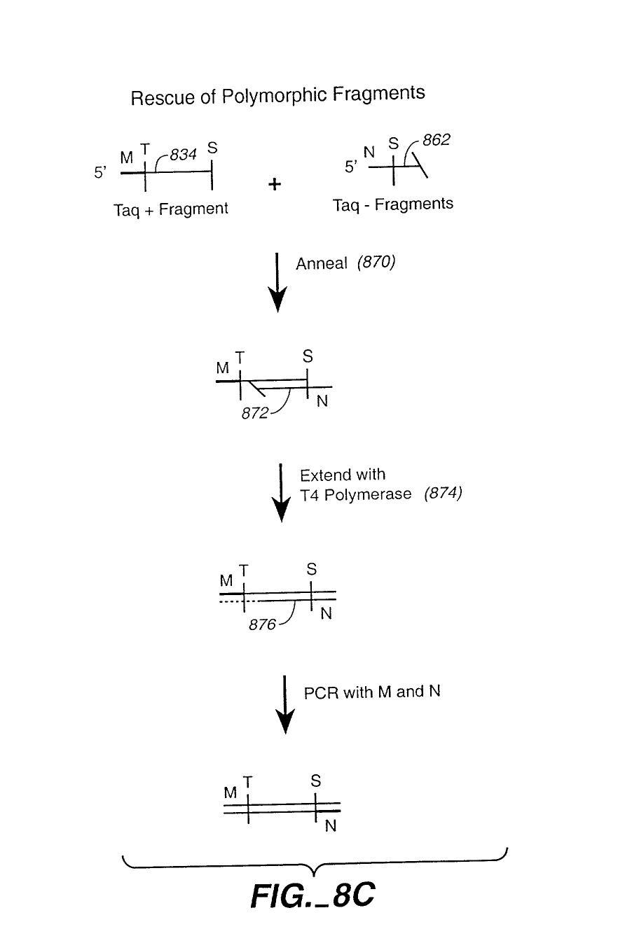

To the mixture of fragments (212) is added M adaptors which are capable of being ligated under conventional reaction conditions to the protruding strands of fragments (211) which have an end produced by cleavage with T. Again this results in a population of at least two kinds of fragments (216): those (213) having a Q adaptor at each end (“Q-Q fragments”), and those (215a and 215b) having a Q adaptor at one end and an M adaptor at the other end (“Q-M fragments”). In those instances where there are multiple t restriction sites within the same fragment “M-M fragments” are formed. If this is the case, as illustrated in FIG. 8A by fragment (812), amplification with M and Q primers eliminates M-M fragments from the mixture because of a 1 base pair gap present on one of the strands of the M-M fragments. The length of the M adaptor is selected as described for the Q adaptor; however, the sequence of the M adaptor is selected to be sufficiently different from that of the Q adaptor so that there is little or no possibility of cross-hybridization between primers during an operation such as PCR. M adaptors further have a 3′ protruding strand at the end distal to the restriction fragment to which it is ligated, so that such strand is not digested by 3′ exonucleases requiring double stranded DNA substrates, such as E. coli exonuclease III.

Alternative means for generating full-length single stranded forms of the Q-M fragment are available, including asymmetric PCR, PCR with one nuclease-resistant primer followed by exonuclease digestion, melting the complements from avidin-captured biotinylated strands, and the like, e.g., Birren et al., editors, Genome Analysis: A Laboratory Manual, Vol. 1 (Cold Spring Harbor Laboratory Press, New York, 1997); Hultman et al., Nucleic Acids Research 17: 4937-4946 (1989); Straus et al., BioTechniques 10: 376-384 (1991); Nikiforow et al., PCR Methods andApplications 3:285-291 (1994); and the like, which references are incorporated by reference.

Returning to FIG. 2B, mixture (216) is digested (218) with a 3′ exonuclease to produce mixture (220) comprising a full length single stranded fragment (217) from each Q-M fragment (215) and two half-length single stranded fragments (219) from each Q-Q fragment (213). To mixture (220) is added (222) primer (224) specific for the primer binding site of M adaptor. After annealing, primer (224) is extended to give double stranded fragment (228), which is then amplified in a PCR using a primer specific to Q adaptor and primer (224) specific for M adaptor. Primer (224) contains several nuclease-resistant linkages at its 5′ end. Preferably, the number of such linkages are in the range of from two to four. Also preferably, the nuclease resistant linkages are phosphorothioate linkages, which may be synthesized using conventional protocols, e.g., Eckstein, editor, Oligonucleotides and Analogues (IRL Press, Oxford, 1991).

Fragments (228) are then cleaved (232) with S to remove the Q adaptor leaving fragments (230), which are then digested with a 5′ 3′ exonuclease to produce a population of single stranded fragments (238). Such 5′ 3′ exonucleases include T7 gene 6 exonuclease (available from United States Biochemical) and may be used in accordance with the protocol of Straus et al. (1991), BioTechniques 10:376-384.

As shown in FIG. 2C, fragments (252) from reaction mixture (204) are processed separately as follows: To fragments (252), N adaptors are ligated using conventional protocols to produce a population (256) of fragments having N adaptors at each end. The length of the N adaptor is selected as described for the Q adaptor; however, the sequence of the N adaptor is selected to be sufficiently different from that of the M adaptor and Q adaptor so that there is little or no possibility of cross hybridization during an operation such as PCR. Fragments of population (256) are then cleaved (258) with T, after which the fragments of the mixture are amplified using primers specific for N; thus, the mixture is greatly enriched in fragments lacking a t restriction site. The amplified fragments are then digested (262) with a 3′ exonuclease, such as E. coli exonuclease III, to give a mixture (266) of single stranded half length fragments (264).

As shown in FIG. 2D, fragments (238) and fragments (266) are combined under conditions that permit complementary strands to hybridize (268). After stable hybrids are formed, repair synthesis is performed on the hybrids to produce double stranded fragments (273), and the double stranded fragments are amplified to form the reference population of restriction fragments with respect to restriction endonucleases S and T.

The nature of the reference library will be influenced by the restriction enzymes and adaptors used to construct the library. For example, reversing the order of restriction enzymes S and T in FIGS. 2A-2D and adding M adaptors that bind to the s restriction site and Q and N adaptors that bind to the t restriction site will result in a reference library corresponding to polymorphisms at restriction site s. A person skilled in the art will also understand that substituting other restriction enzymes for S and T will produce fragments with different protruding ends at different sites within the nucleic acid pool. This will result in a reference library made up of fragments from different polymorphic subregions which are specifically defined by the restriction endonucleases used.

Whenever the method of the invention is applied to populations of DNA that include all or a substantial fraction of complete genomes, particularly mammalian or higher plant genomes, the step of forming hybrids may include a step of forming subpopulations of DNA in order to reduce the complexity of the DNA populations prior to hybridization. As used herein, the term “complexity” in reference to a population of polynucleotides means the number of different species of polynucleotides present in the population. For example, nucleic acid pools may be treated to reduce the complexity of DNA populations using differential PCR amplification using sets of primers having different 3′-terminal nucleotides, e.g., Pardee et al., U.S. Pat. No. 5,262,311; amplification after ligation of indexing linkers, e.g., Kato, U.S. Pat. No. 5,707,807; Deugau et al., U.S. Pat. No. 5,508,169; and Sibson, U.S. Pat. No. 5,728,524; and the like, which references are incorporated by reference. Other ways of reducing complexity include pre-treating DNA to remove repetitive sequences.

Repeated sequences are dispersed throughout eucaryotic genomes. See Davidson and Britten (1973) The Quarterly Review of Biology 48:565-613; Britten and Davidson (1971) The Quarterly Review of Biology 46:111-138. In humans, repeated sequences are found at intervals of a few thousand base pairs throughout at least 80% of the genome. See Sealey, et al., (1985) Nuc. Acid Res. 13:1905-1923. Thus, the reference library may be skewed by the presence of such repetitive elements. Such repetitive sequences may affect the polymorphic sequences present in the reference libraries because of cross hybridization which may occur between shared repetitive elements in other parts of the genome during library formation. This problem may be substantially reduced by pre-treating genomic DNA to form subpopulations of genomic DNA enriched for non-repetitive sequences.

By “repetitive sequences” herein is meant nucleotide sequences which are repeated many times and reassociate at Cot values lower than expected from the genome size (Lin and Lee (1981) Biochimica et Biophysica Acta, 653:193-203).

Nucleic acid pools may be treated to form subpopulations of DNA depleted in repetitive sequences before or during the making of the reference library. Preferably 10% of the repetitive sequences are removed. More preferably 25% of the repetitive sequences are removed. Even more preferably 50% of the repetitive sequences are removed. Further reductions in repetitive sequences also may be desirable, including removal of 75% to 90% of the repetitive sequences present in the starting nucleic acid pool.

Subpopulations depleted in repetitive sequence may be formed using methods which rely on the higher effective hybridization rate of complementary nucleic acid sequences which are present at higher concentration. Thus, if a heterogeneous mixture of nucleic acid fragments is denatured and incubated under conditions that permit hybridization, those sequences present at relatively high concentrations, e.g., repetitive sequences, will become double stranded more rapidly than those present at lower concentrations. The double stranded molecules are separated from the single stranded molecules using methods well known to those of ordinary skill in the art.

Accordingly, subpopulations of DNA enriched for non-repetitive DNA may be obtained by pre-treating genomic nucleic acid pools. As used herein, “non-repetitive” DNA is DNA other than repetitive DNA. Non-repetitive DNA reassociates at Cₒt values consistent with genome size and includes single- and low-copy DNA sequences. “Single” and “low-copy” DNA sequences are defined herein as sequences which occur relatively rarely in eucaryotic genomes. Cₒt is the molar concentration of DNA multiplied by the time allowed for resassociation in a given solvent. Lin and Lee (1981) Biochimica et Biophysica Acta 653:193-203.

In a preferred embodiment, subpopulations of non-repetitive DNA are formed by pre-treating pooled genomic DNA to remove repetitive sequences. For example, pooled genomic DNA is digested, denatured and then allowed to reassociate for a short period of time. The formation of double stranded repetitive DNA sequences is kinetically favored over more unique sequences. See Lin and Lee (1981) Biochimica et Biophysica Acta 653:193-203. The addition of a nuclease, such as exonuclease III, that can act upon double stranded molecules may deplete or eliminate the double stranded repetitive sequences present in the reaction mixture. Following treatment with the nuclease, the remaining sequences are amplified, thereby forming a subpopulation of nucleic acid fragments enriched for non-repetitive DNA. Adaptors, i.e., Q, N or M, may be added before or after treatment with the nuclease so that the remaining sequences can be amplified.

Alternatively, double stranded repetitive sequences can be removed using hydroxyapatite columns. Single and double stranded nucleic acid molecules have different binding characteristics to hydroxyapaptite. Using methods which rely on these differences, the fraction of genomic DNA containing repetitive sequences can be separated from non-repetitive DNA by denaturing genomic DNA, allowing it to reassociate under appropriate conditions to a particular Cₒt value, followed by separation of the double stranded molecules which bind to hydroxyapatite. See Gray et al., U.S. Pat. No. 5,756,696 (issued May 26, 1998); Current Protocols in Molecular Biology (1997) 2.13.1-2.13.3; Soares, et al., (1994) Proc. Natl. Acad. Sci. USA, 91:9228-9232; Ko (1990) Nuc. Acid Res. 18:5705; Kantor and Schwartz (1979) Anal. Biochemistry 97:77-84.

Other approaches useful for removing repetitive DNA sequences include magnetic purification and PCR-assisted affinity chromatography (Craig, et al, (1997) Hum. Genet. 100:472-476; Durm et al., (1998) BioTechniques 24:820-825); single stranded “absorbing” DNA attached to a solid support (Brison, et al. (1982) Molecular and Cellular Biology 2:578-587; and the use of hybridization probes representative of highly repeated sequence families (Sealy, et al. (1985) Nuc. Acids Res. 13:1905-1923; Wetmur (1991) Critical Reviews in Biochemistry and Molecular Biology 26:227-259).

Alternatively, subpopulations of nucleic acid fragments enriched for non-repetitive DNA can be formed by denaturing pooled genomic DNA and reassociating over a long period of time. This approach favors the formation of D-loops in repetitive DNA duplexes, whereas stable duplexes are formed between complementary sequences of non-repetitive DNA. Addition of single strand-specific endonucleases, such as nuclease S1, results in the removal of repetitive sequences which have formed a D-loop from the mixture, thereby enriching for non-repetitive DNA sequences. See Wetmur, (1991) Critical Reviews in Biochemistry and Molecular Biology 26:227-259.

In a preferred embodiment, repetitive DNA sequences are removed during the making of the reference library using single strand endonucleases. By “single strand endonuclease” herein is meant any endonuclease which recognizes and cleaves non-perfectly matched DNA-DNA or DNA-RNA duplexes, cruciform DNA structures, Holliday structures or junctions, heteroduplex DNA, and nicked double-stranded DNA. Preferred single strand endonucleases include T7 endonuclease I, mung bean nuclease and S1 nuclease. In a preferred embodiment, T7 endonuclease I is used.

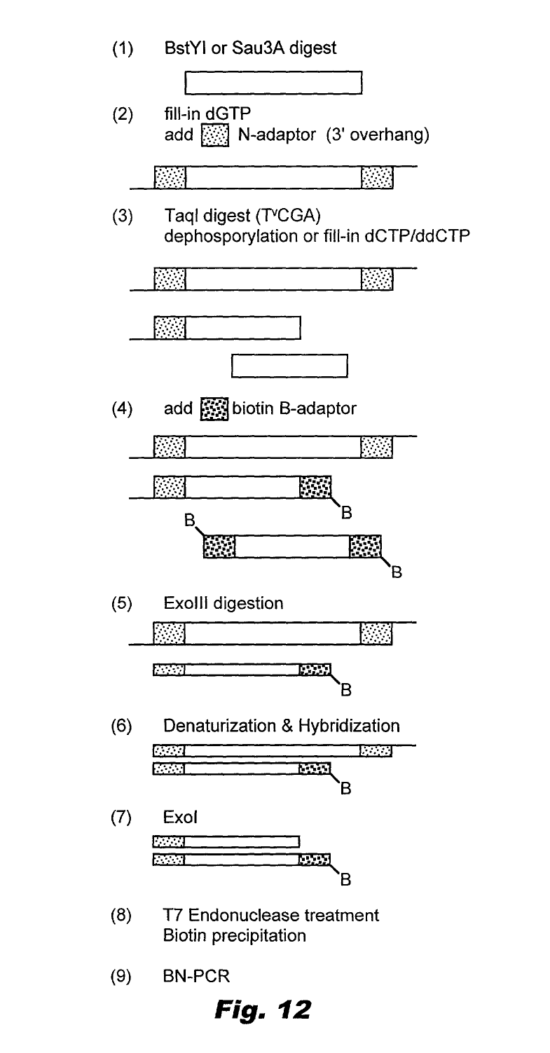

In a preferred embodiment, a heterogeneous mixture of nucleic acid fragments is obtained when digested with at least two restriction enzymes. As shown in FIG. 2A, DNA is cleaved with a first restriction endonuclease to produce a population of restriction fragments, i.e., restriction fragments (204). In place of the Q adaptor, an Exo III resistant linker “N” is ligated in a conventional ligation reaction to give a fragment-Exo III resistant linker complex (first ligation product). See FIG. 12. By “Exo III resistant” linker herein is meant a linker which comprises a 3′-overhang (See FIG. 12, step 3).

The first ligation product is then digested with a second restriction endonuclease to yield restriction fragments similar to fragments (212) illustrated in FIG. 2A. To this mixture is added Exo III susceptible linkers (“B” in FIG. 12) which are capable of being ligated under conventional reaction conditions to the ends of fragments produced by cleavage with the second restriction endonuclease. This forms a second ligation product population which includes structures corresponding to the first ligation product (FIG. 12, step 4). By “Exo III susceptible” herein is meant linkers which comprise either a 5′-overhang or a blunt end. The second ligation product population is then digested with Exo III, to form a third ligation product population comprising (i) single stranded DNA comprising end sequences corresponding to said Exo III resistant and Exo m susceptible linkers and (ii) double stranded DNA comprising end sequences corresponding to said Exo III resistant linkers, and denatured. See FIG. 12, step 5.

The resultant mixture is incubated under conditions that permit hybridization, such that a first population of duplexes (reannealed third ligation product population) is formed. Preferably, the first duplex population is treated with a single strand endonuclease for a sufficient time to digest mismatched duplexes. The resulting mixture is denatured and reannealed, such that a second population of complementary duplexes is formed. A reference population of restriction fragments is then isolated from the second population of duplexes.

In one embodiment, purification of isolated duplexes from the second population of duplexes may be done using the members of binding pairs, i.e., biotin-streptavidin. Biotinylated nucleic acid fragments are formed using a biotin-Exo II susceptible linker (See FIG. 12, step 4). Biotinylated molecules are isolated following single strand endonuclease treatment using streptavidin-coupled beads, columns, etc. (FIG. 12, steps 8 and 9; Li, J., et al. (2000) Nucleic Acids Research 28:i-v).

Once made, the reference libraries find use in a variety of applications. Generally, the reference libraries are used to compare the frequency of various polymorphisms in a population of interest. Polymorphisms which occur more frequently in one population than another, can be isolated and identified using the methods of the invention. When used to analyze other populations, a pool of DNA from individuals having a first phenotype is compared to a population which demonstrates a second phenotype.

Accordingly, the reference libraries of the invention can be used to screen for polymorphic markers in close proximity to genes which may be associated with one or more phenotypes or genotypes. An advantage to using the reference libraries to screen for polymorphic markers associated with a phenotype or genotype is that prior knowledge of the trait is not required. Thus, polymorphisms associated with genotypes which show simple Mendialian inheritance, as well as genotypes or phenotypes associated with a complex trait can be detected using the compositions and methods of the present invention. For example, response to medication, a complex trait governed by a number of genes, is amenable to this type of approach. In particular, this approach can be used to identify those individuals likely to benefit from new medications being developed and those likely to suffer adverse side-effects.

Other phenotypes of biological interest which can be screened using polymorphic probes include common diseases in humans such as cardiovascular diseases, autoimmune diseases, cancer, diabetes, schizophrenia, bipolar disorder and other psychiatric disorders. See Kwok and Gu (1999) Mol Medicine Today 5:538; Risch and Merikangas (1996) Science 273:1516; Landu and Schork (1994) Science 265:2037. In addition, polymorphisms in other organisms, i.e., plants, associated with phenotypical traits such as disease resistance and yield can also be screened using various embodiments of the invention. See Kesseli et al. (1994) Genetics 136:1435; Michelmore et al. (1991) Genetics 88:9828.

Generally, the frequency of polymorphisms in a population of interest is compared as follows. A pool of DNA from individuals having a first phenotype is digested with a first restriction endonuclease to form a pool of restriction fragments. Fragments lacking the polymorphism are then selected. A second pool of DNA from individuals having a second phenotype is similarly treated to select for subregions which also lack the polymorphism. The reference library is then contacted with the fragments which lack the polymorphism and the relative frequency of the polymorphic subregions in the individuals which lack the polymorphism is determined.

The pools from the two populations may be analyzed separately or mixed together and analyzed. The frequency of the polymorphism in the two populations may be determined by labeling the fragments in the two pools. The label can be the same if the two pools are analyzed separately or different labels can be used to distinguish the fragments from the two populations if the pools are mixed. As explained in more detail hereinafter, labels suitable for use include light generating labels such as fluorescent dyes.

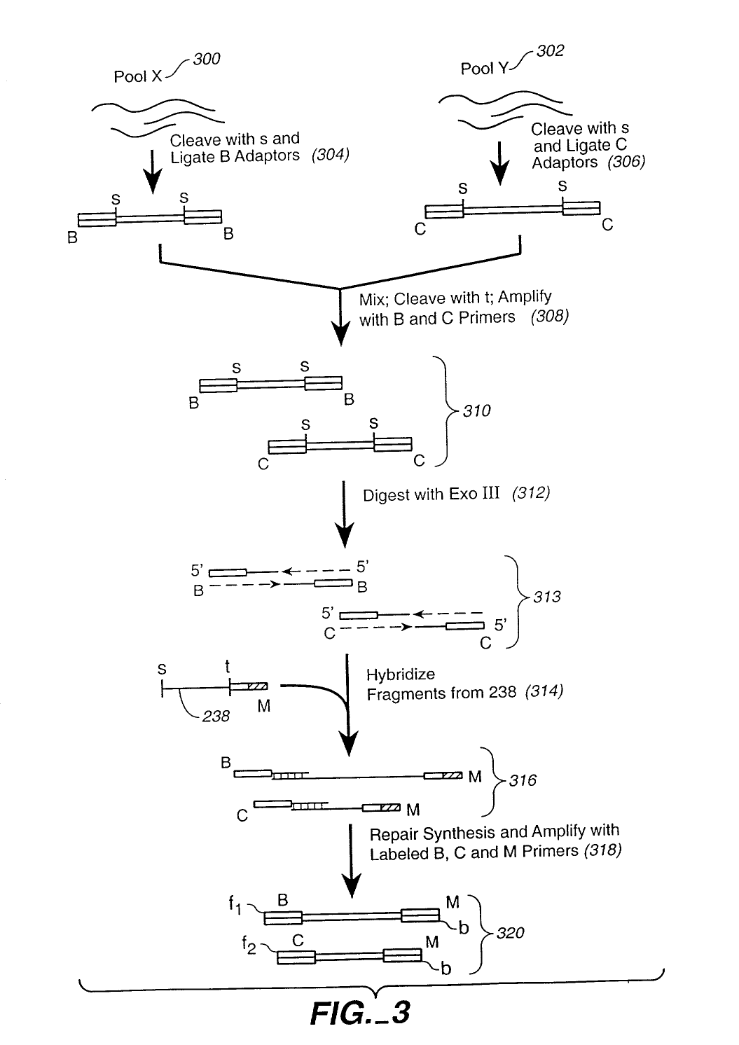

A preferred method for the use of the reference library is set forth in FIG. 3. Genomic DNA is exacted from individuals of a first (300) and second (302) pool of individuals, designated X and Y, respectively, in FIG. 3. Preferably, equal amounts of DNA are contributed from each individual. DNA from pool X is cleaved (304) with restriction endonuclease S and B adaptors are ligated to the ends of the resulting fragments. B adaptors are selected as described above for the Q adaptors. Separately, DNA from pool Y is cleaved (306) with restriction endonuclease S and C adaptors are ligated to the ends of the resulting fragments. C adaptors are selected as described above for the Q adaptors. As with the Q adaptors, the B and C adaptors contain primer binding sites for later amplification by PCR. The sequences selected for these primer binding sites should be sufficiently different that there is little or no cross hybridization of the respective primers. Equal amounts of adaptor-fragment complexes from reactions (304) and (306) are combined, after which the complexes are cleaved with restriction endonuclease T, followed by amplification using both B- and C-specific primers in a conventional PCR. This gives a population (310) of adaptor-fragment complexes that lack internal t restriction sites. Population (310) is digested (312) with a 3′ exonuclease, e.g., E. coli exonuclease III, to give half-length fragments (313), which are then hybridized (314) with fragments (238) to form hybrids (316). Repair synthesis (318) is carried out on hybrids (316), after which the resulting fragments are amplified using primers specific for the primer binding sites of the B, C, and M adaptors.

Preferably, the respective primers carry distinguishable labels, e.g., fluorescent labels, by which relative numbers of fragments from the two pools are compared by competitive hybridization to complementary strands from the reference population attached to solid phase supports. The results of such amplification are illustrated as fragments (320) wherein the primers specific for B adaptors carry fluorescent label f1, primers specific for C adaptors carry fluorescent label f₂, and primers specific for M adaptors carry a biotin, indicated by “b” for purifying the fragments from the reaction mixture. As suggested by fragments (320) in FIG. 3, single stranded labeled probes may be derived from fragments (320) by isolating the fragments via a solid phase avidinated support, followed by melting of the non-covalently attached strands carrying the fluorescent labels.

The skilled artisan will understand that a similar analysis can be made by selecting for t⁺ restriction sites in first and second populations by adapting the protocol referred to in FIG. 3. As in FIG. 3, pools X and Y are cleaved with restriction enzyme S. Fragments from pool X are ligated with B adaptors, and fragments from pool Y are ligated with C adaptors. The fragments are then cleaved with T and ligated with M adaptors. To eliminate t⁻ fragments, the mixture is first treated with exonuclease III. Following exonuclease III treatment, t⁺ fragments are amplified using B and M primers. This selects for t⁺ DNA which is then analyzed with the reference library as described above.

Once made, the reference libraries or polymorphic probes may be attached to solid phase supports either directly or via oligonucleotide tags or tag complements (described more fully below). Solid phase supports for use with the reference libraries may have a wide variety of forms, including microparticles, beads, membranes, slides, plates, micromachined chips, and the like. Likewise, solid phase supports may comprise a wide variety of compositions, including glass, plastic, silicon, alkanethiolate-derivatized gold, cellulose, low cross-linked and high cross-linked polystyrene, silica gel, polyamide, and the like.

Identical copies of the same sequence (i.e., polymorphic probes) from the reference library may be attached to discrete particles to form subpopulations of miroparticles. A multiplicity of such subpopulations where each subpopulation contains different polymorphic probes forms a reference library composition which may be used to test other populations. Alternatively, identical copies of the same sequence may be attached to single or multiple supports such that spatially discrete regions each containing the same sequence of different polymorphic probes is formed. In the latter embodiment, the area of the regions may vary according to particular applications; usually, the regions range in area from several μm², e.g., 3-5, to several hundred μm², e.g., 100-500. Preferably, such regions are spatially discrete so that signals generated by events, e.g., fluorescent emissions, at adjacent regions can be resolved by the detection system being employed.

In a preferred embodiment, arrays having defined regions on the surface of solid phase supports can be formed using the polymorphic probes of the invention. Methods for creating such arrays include, but are not limited to: (1) using pins to distribute preformed nucleic acid solutions in defined regions (Brown and Botstein, (1999) Nature Genet. 21(Suppl.):33; Duggan et al., (1999) Nature Genet. 21(Suppl.):10; McAllister, et al, (1997) Am. J. Hum. Genet. 21(Suppl):¹³⁸⁷; Schena, et al, (1995) Science 270:467); (2) using a capillary dispenser to place the reference libraries in defined regions on a solid support (see International Application No. PCT/US95/07659); (3) using ink-jet techniques in which oligonucleotides are synthesized base by base through sequential solution-based reactions on a solid surface (Blanchard, et al., (1996) Biosens. and Bioelectron. 11:687); (4) synthesizing the oligonucleotides tags directly onto the surface of a solid support using patterned light-directed combinatorial chemical synthesis and using the tags to sort polymorphic probes conjugated to tag complements into defined regions (see Fodor et al., U.S. Pat. No. 5,744,305 issued Apr. 28, 1998; Chee et al., U.S. Pat. No. 5,837,832 issued Nov. 17, 1998; Fodor, (1997) Science 277:393); and (5) by attaching oligonucleotides to microspheres for preparing fiber-optic arrays (Walt, et al., International Application No. PCT/US98/09163).

For use in hybridization reactions, identical copies of fragments from the reference library, i.e., referred to herein as “clonal subpopulations,” are attached to one or more solid phase supports in separate regions so that the fragments may be employed in hybridization assays. The construction of such hybridization supports may be carried out in a variety of ways. For example, the fragments may be amplified by PCR or by cloning in a vector. By “vector” or “cloning vector” or grammatical equivalents herein is meant an extrachromosomal genetic element which can be used to replicate a DNA fragment in a host organism. A wide variety of cloning vectors are commercially available for use with the invention, e.g., New England Biolabs (Beverly, Mass.); Stratagene Cloning Systems (La Jolla, Calif.); Clontech Laboratories (Palo Alto, Calif.); and the like.

In a preferred embodiment, the nucleic acid fragments of the invention are cloned in bacterial vectors. In such a case, bacterial colonies may be formed and individual clones picked for further amplification and attachment to either planar arrays or microparticles. Technology for carrying out such operations are well known, e.g., Brown et al., U.S. Pat. No. 5,807,522; Ghosh et al., U.S. Pat. No. 5,478,893; Fodor et al., U.S. Pat. Nos. 5,445,934; 5,744,305; 5,800,992; and the like. The number of copies of a fragment in a clonal subpopulation may vary widely in different embodiments depending on several factors, including the density of tag complements on the solid phase supports, the size and composition of microparticles used, the duration of hybridization reaction, the complexity of the tag repertoire, the concentration of individual tags, the tag-fragment sample size, the labeling means for generating optical signals, the particle sorting means, signal detection system, and the like. Guidance for making design choices relating to these factors is readily available in the literature on flow cytometry, fluorescence microscopy, molecular biology, hybridization technology, and related disciplines, as represented by the references cited herein.

Preferably, the number of copies of a fragment in a clonal subpopulation is sufficient to permit fluorescence-activated cell sorter (“FACS”) sorting of microparticles, wherein fluorescent signals are generated by one or more fluorescent dye molecules carried by the fragments attached to the microparticles. Typically, this number can be as low as a few thousand, e.g., 3-5,000, when a fluorescent molecule such as fluorescein is used, and as low as several hundred, e.g., 800-8000, when a rhodamine dye, such as rhodamine 6G, is used. More preferably, when loaded microparticles are sorted by FACS, clonal subpopulations consist of at least 10⁴ copies of a fragment; and most preferably, in such embodiments, clonal subpopulations consist of at least 10⁵ copies of a fragment.

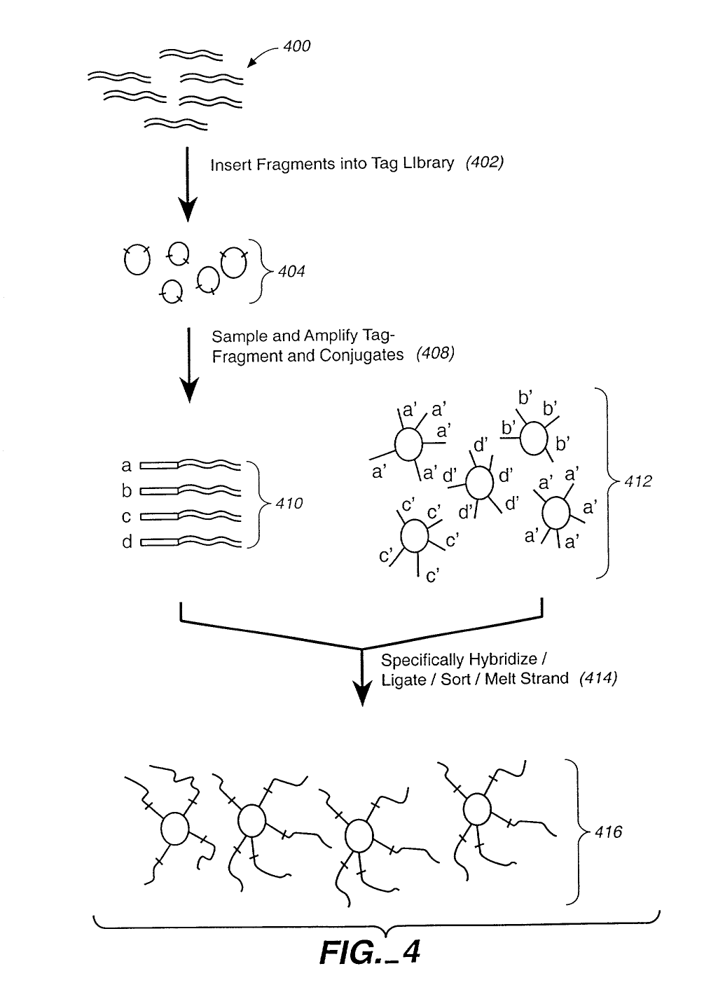

Briefly, as summarized (274) in FIG. 2D and illustrated more fully in FIG. 4, oligonucleotide tags from a large repertoire (404) are attached (402) to the fragments (400) to form tag-fragment conjugates, a sample of tag-fragment conjugates is taken so that substantially all different fragments have different tags, the sample of tag-fragment conjugates is amplified (408), and the amplified copies (410) are specifically hybridized (414) to one or more solid phase supports (412). Preferably, the one or more solid phase supports is a population of microparticles (412) carrying oligonucleotides with complementary sequences to the tags of the tag-fragment conjugates. In the preferred embodiment employing microparticles, after specific hybridization, tag-fragments conjugates are ligated to the tag complements attached to the microparticles and the non-covalently attached strand is melt off giving microparticles (416) which are ready to accept the hybridization probes described below.

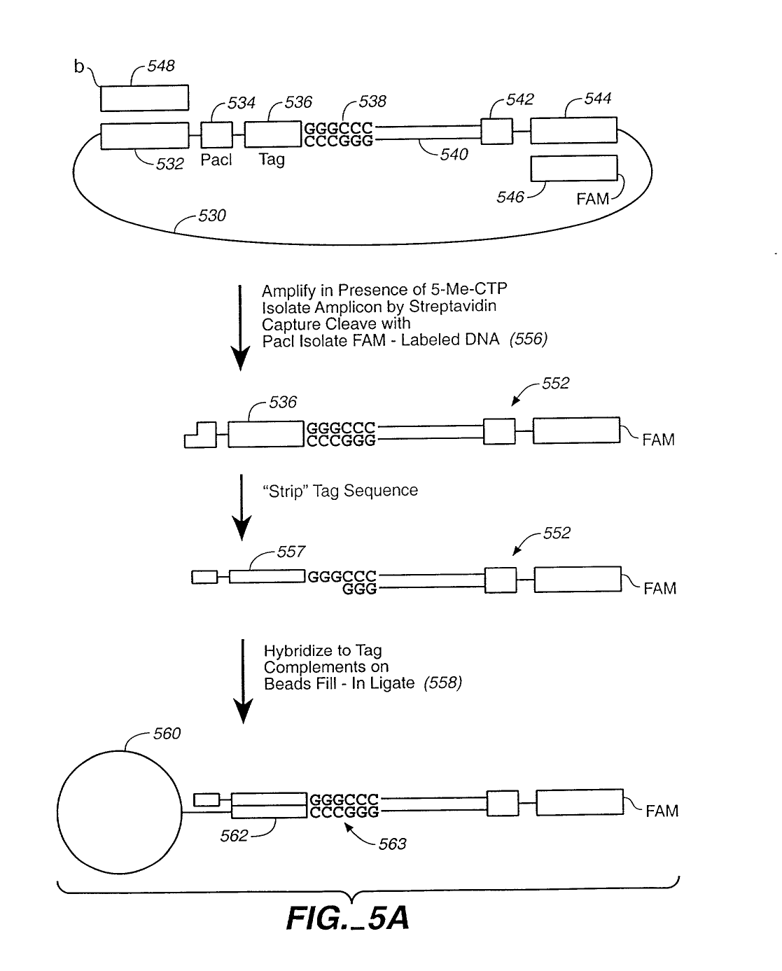

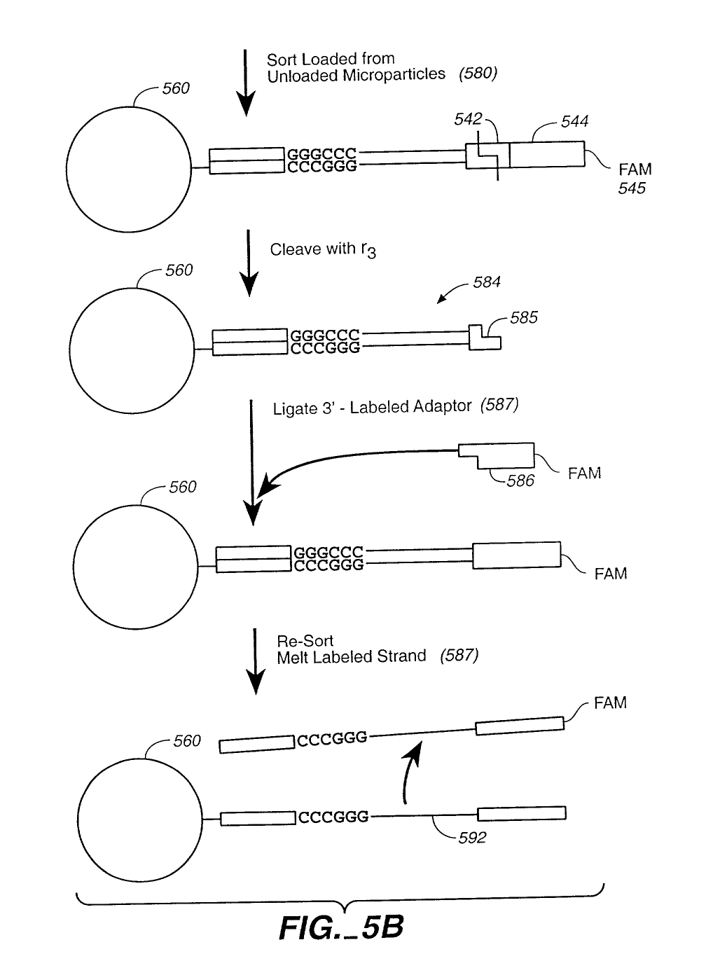

A preferred method of attaching oligonucleotide tags to fragments is further illustrated in FIGS. 5A and 5B. Preferably, fragments are inserted into vector (530) which after insertion comprises the following sequence of elements: first primer binding site (532), restriction site r₁ (534), oligonucleotide tag (536), junction (538), fragment (540), restriction site r₂ (542), and second primer binding site (544). After a sample is taken of the vectors containing tag-fragment conjugates the following steps are implemented: The tag-fragment conjugates are preferably amplified from vector (530) by use of biotinylated primer (548) and labeled primer (546) in a conventional polymerase chain reaction (PCR) in the presence of 5-methyldeoxycytidine triphosphate, after which the resulting amplicon is isolated by streptavidin capture. As used herein, “amplicon” means the product of an amplification reaction. That is, it is a population of polynucleotides, usually double stranded, that are replicated from a few starting sequences. Amplicons may be produced in a polymerase chain reaction or by replication in a cloning vector. To release the captured amplicon from a support with minimal probability of cleavage occurring at a site internal to the fragment of the amplicon, restriction site r₁ preferably corresponds to a rare-cutting restriction endonuclease, such as Pac I, Not I, Fse I, Pme I, Swa I, or the like. Junction (538) which is illustrated as the sequence:

5′. . . GGGCCC . . .
3′. . . CCCGGG . . .

causes the DNA polymerase “stripping” reaction to be halted at the G triplet, when an appropriate DNA polymerase is used with dGTP. Briefly, in the “stripping” reaction, the 3′ 5′ exonuclease activity of a DNA polymerase, preferably T4 DNA polymerase, is used to render the tag of the tag-fragment conjugate single stranded, as taught by Brenner, U.S. Pat. No. 5,604,097; and Kuijper et al. (1992) Gene 112: 147-155 (1992).

In the preferred embodiment where sorting is accomplished by formation of duplexes between tags and tag complements, tags of tag-fragment conjugates are rendered single stranded by first selecting words that contain only three of the four natural nucleotides, and then by preferentially digesting the three nucleotide types from the tag-fragment conjugate in the 3′0 5′ direction with the 3′ 5′ exonuclease activity of a DNA polymerase.

In the preferred embodiment, oligonucleotide tags are designed to contain only A's, G's, and T's; thus, tag complements (including that in the double stranded tag-fragment conjugate) consist of only A's, C's, and T's. When the released tag-fragment conjugates are treated with T4 DNA polymerase in the presence of dGTP, the complementary strands of the tags are “stripped” away to the first G. At that point, the incorporation of dG by the DNA polymerase balances the exonuclease activity of the DNA polymerase, effectively halting the “stripping” reaction. From the above description, it is clear that one of ordinary skill could make many alternative design choices for carrying out the same objective, i.e., rendering the tags single stranded. Such choices could include selection of different enzymes, different compositions of words making up the tags, and the like.

When the “stripping” reaction is quenched, the result is duplex (552) with single stranded tag (557). After isolation, steps (558) are implemented: the tag-fragment conjugates are hybridized to tag complements attached to microparticles, a fill-in reaction is carried out to fill any gap between the complementary strand of the tag-fragment conjugate and the 5′ end of tag complement (562) attached to microparticle (560), and the complementary strand of the tag-fragment conjugate is covalently bonded to the 5′ end (563) of tag complement (562) by treating with a ligase. This embodiment requires, of course, that the 5′ end of the tag complement be phosphorylated, e.g., by a kinase, such as, T4 polynucleotide kinase, or the like. The fill-in reaction is preferably carried out because the “stripping” reaction does not always halt at the first G. Preferably, the fill-in reaction uses a DNA polymerase lacking 5′ 3′ exonuclease activity and strand displacement activity, such as T4 DNA polymerase. Also preferably, all four dNTPs are used in the fill-in reaction, in case the “stripping” extended beyond the G triplet.

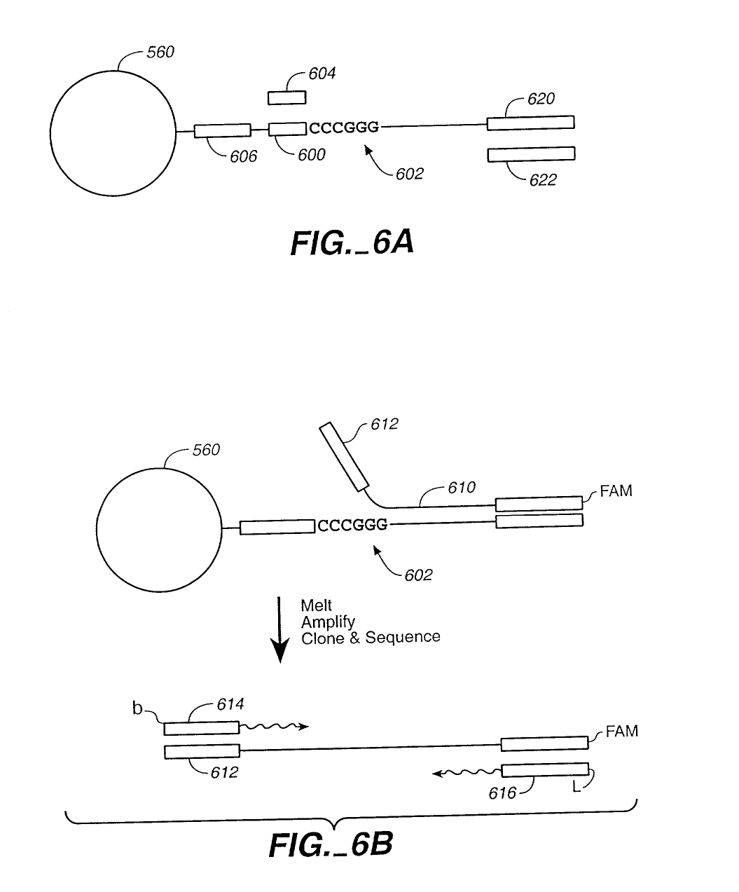

As explained further below, the tag-fragment conjugates are hybridized to the fill repertoire of tag complements. That is, among the population of microparticles, there are microparticles having every tag sequence of the entire repertoire. Thus, the tag-fragment conjugates will hybridize to tag complements on only about one percent of the microparticles. Microparticles to which tag-fragments have been hybridized are referred to herein as “loaded microparticles.” For greater efficiency, loaded microparticles are preferably separated from unloaded microparticles for further processing. Such separation is conveniently accomplished by use of a FACS, or similar instrument that permits rapid manipulation and sorting of large numbers of individual microparticles. In the embodiment illustrated in FIG. 6A, a fluorescent label, e.g., FAM (a fluorescein derivative, Haugland, Handbook of Fluorescent Probes and Research Chemicals, Sixth Edition, (Molecular Probes, Eugene, Ore. 1996)) is attached by way of primer (546).

As shown in FIG. 6B, after FACS, or like sorting (580), loaded microparticles (560) are isolated, treated to remove label (545), and treated to melt off the non-covalently attached strand. Label (545) is removed or inactivated so that it does not interfere with the labels of the competitively hybridized strands. Preferably, the tag-fragment conjugates are treated with a restriction endonuclease recognizing site r₃ (542) which cleaves the tag-fragment conjugates adjacent to primer binding site (544), thereby removing label (545) carried by the “bottom” strand, i.e., the strand having its 5′ end distal to the microparticle. Preferably, this cleavage results in microparticle (560) with double stranded tag-fragment conjugate (584) having protruding strand (585). 3′-labeled adaptor (586) is then annealed and ligated (587) to protruding strand (585), after which the loaded microparticles are re-sorted by means of the 3′-label. The strand carrying the 3′-label is melted off to leave a covalently attached single strand of the fragment (592) ready to accept probes, produced as illustrated in FIG. 4. Preferably, the 3′-labeled strand is melted off with sodium hydroxide treatment, or treatment with like reagent.

An important feature of the invention is the use of oligonucleotide tags which are members of a minimally cross-hybridizing set of oligonucleotides to construct reference DNA populations attached to solid phase supports, preferably microparticles.

The term “oligonucleotide” as used herein includes linear oligomers of natural or modified monomers or linkages, including deoxyribonucleosides, ribonucleosides, and the like, capable of specifically binding to a target polynucleotide by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g., 3-4, to several tens of monomeric units, e.g., 40-60. Whenever an oligonucleotide is represented by a sequence of letters, such as “ATGCCTG,” it will be understood that the nucleotides are in 5′→3′ order from left to right and that “A” denotes deoxyadenosine, “C” denotes deoxycytidine, “G” denotes deoxyguanosine, “T” denotes thymidine, and “U” denotes uridine, unless otherwise noted. The term “dNTP” is an abreviation for “a deoxyribonucleoside triphosphate,” and “dATP”, “dCTP”, “dGTP”, “dTTP”, and “dUTP” represent the triphosphate derivatives of the individual deoxyribonucleosides. Usually oligonucleotides comprise the natural nucleotides; however, they may also comprise non-natural nucleotide analogs. It is clear to those skilled in the art when oligonucleotides having natural or non-natural nucleotides may be employed, e.g., where processing by enzymes is called for, usually oligonucleotides consisting of natural nucleotides are required.

“Perfectly matched” in reference to a duplex means that the poly- or oligonucleotide strands making up the duplex form a double stranded structure with one other such that every nucleotide in each strand undergoes Watson-Crick base pairing with a nucleotide in the other strand. The term also comprehends the pairing of nucleoside analogs, such as deoxyinosine, nucleosides with 2-aminopurine bases, and the like, that may be employed. In reference to a triplex, the term means that the triplex consists of a perfectly matched duplex and a third strand in which every nucleotide undergoes Hoogsteen or reverse Hoogsteen association with a basepair of the perfectly matched duplex.

By “mismatch” herein is meant a base pair between any two of the bases A, T, (or U for RNA), G and other than the Watson-Crick base pairs G-C, and A-T. The eight possible mismatches are A-A, T-T, G-G, C-C, T-G, C-A, T-C and A-G.

The sequences of oligonucleotides of a minimally cross-hybridizing set differ from the sequences of every other member of the same set by at least two nucleotides. Thus, each member of such a set cannot form a duplex (or triplex) with the complement of any other member with less than two mismatches. Complements of oligonucleotide tags, referred to herein as “tag complements,” may comprise natural nucleotides or non-natural nucleotide analogs. When oligonucleotide tags are used for sorting, as is the case for constructing a reference DNA population, tag complements are preferably attached to solid phase supports. Oligonucleotide tags when used with their corresponding tag complements provide a means of enhancing specificity of hybridization for sorting, tracking, or labeling molecules, especially polynucleotides, such as cDNAs or mRNAs derived from expressed genes.

Minimally cross-hybridizing sets of oligonucleotide tags and tag complements may be synthesized either combinatorially or individually depending on the size of the set desired and the degree to which cross-hybridization is sought to be minimized (or stated another way, the degree to which specificity is sought to be enhanced). For example, a minimally cross-hybridizing set may consist of a set of individually synthesized 10-mer sequences that differ from each other by at least 4 nucleotides, such set having a maximum size of 332, when constructed as disclosed in Brenner et al., International patent application PCT/US96/09513. Alternatively, a minimally cross-hybridizing set of oligonucleotide tags may also be assembled combinatorially from subunits which themselves are selected from a minimally cross-hybridizing set. For example, a set of minimally cross-hybridizing 12-mers differing from one another by at least three nucleotides may be synthesized by assembling 3 subunits selected from a set of minimally cross-hybridizing 4-mers that each differ from one another by three nucleotides. Such an embodiment gives a maximally sized set of 9³, or 729, 12-mers.

When synthesized combinatorially, an oligonucleotide tag preferably consists of a plurality of subunits, each subunit consisting of an oligonucleotide of 3 to 9 nucleotides in length wherein each subunit is selected from the same minimally cross-hybridizing set. In such embodiments, the number of oligonucleotide tags available depends on the number of subunits per tag and on the length of the subunits.

In a preferred embodiment, the oligonucleotide tags comprise oligonucleotides of the form:

S₁S₂S₃ . . . Sₙ

As used herein, “S₁ through Sₙ” refer to the subunits comprising an oligonucleotide tag having a length from 3 to 9 nucleotides and are selected from a minimally cross hybridizing set. “n” is in the range from 4 to 10, and the overall length of the tag may range from 12 to 60 nucleotides.

Complements of oligonucleotide tags attached to one or more solid phase supports are used to sort polynucleotides from a mixture of polynucleotides each containing a tag. Such tag complements are synthesized on the surface of a solid phase support, such as a microparticle or a specific location on an array of synthesis locations on a single support, such that populations of identical, or substantially identical, sequences are produced in specific regions. That is, the surface of each support, in the case of a bead, or of each region, in the case of an array, is derivatized by copies of only one type of tag complement having a particular sequence. The population of such beads or regions contains a repertoire of tag complements each with distinct sequences. As used herein in reference to oligonucleotide tags and tag complements, the term “repertoire” means the total number of different oligonucleotide tags or tag complements that are employed for solid phase cloning (sorting) or identification. A repertoire may consist of a set of minimally cross-hybridizing set of oligonucleotides that are individually synthesized, or it may consist of a concatenation of oligonucleotides each selected from the same set of minimally cross-hybridizing oligonucleotides. In the latter case, the repertoire is preferably synthesized combinatorially.

Preferably, tag complements are synthesized combinatorially on microparticles, so that each microparticle has attached many copies of the same tag complement. A wide variety of microparticle supports may be used with the invention, including microparticles made of controlled pore glass (CPG), highly cross-linked polystyrene, acrylic copolymers, cellulose, nylon, dextran, latex, polyacrolein, and the like, disclosed in the following exemplary references: Meth. Enzymol., Section A, pages 11-147, vol. 44 (Academic Press, New York, 1976); U.S. Pat. Nos. 4,678,814; 4,413,070; and 4,046;720; and Pon, Chapter 19, in Agrawal, editor, Methods in Molecular Biology, Vol. 20, (Humana Press, Totowa, N.J., 1993). Microparticle supports further include commercially available nucleoside-derivatized CPG and polystyrene beads (e.g., available from PE Applied Biosystems, Foster City, Calif.); derivatized magnetic beads; polystyrene grafted with polyethylene glycol (e.g., TentaGelJ, Rapp Polymere, Tubingen Germany); and the like. Microparticles may also consist of dendrimeric structures, such as disclosed by Nilsen et al, U.S. Pat. No. 5,175,270. Generally, the size and shape of a microparticle is not critical; however, microparticles in the size range of a few, e.g., 1-2, to several hundred, e.g., 200-1000 μm diameter are preferable, as they facilitate the construction and manipulation of large repertoires of oligonucleotide tags with minimal reagent and sample usage. Preferably, glycidal methacrylate (GMA) beads available from Bangs Laboratories (Carmel, Ind.) are used as microparticles in the invention. Such microparticles are useful in a variety of sizes and are available with a variety of linkage groups for synthesizing tags and/or tag complements. More preferably, 5 μm diameter GMA beads are employed.

Polynucleotides to be sorted, or cloned onto a solid phase support, each have an oligonucleotide tag attached, such that different polynucleotides have different tags. This condition is achieved by employing a repertoire of tags substantially greater than the population of polynucleotides and by taking a sufficiently small sample of tagged polynucleotides from the full ensemble of tagged polynucleotides. After such sampling, when the populations of supports and polynucleotides are mixed under conditions which permit specific hybridization of the oligonucleotide tags with their respective complements, identical polynucleotides sort onto particular beads or regions. Of course, the sampled tag-polynucleotide conjugates are preferably amplified, e.g., by polymerase chain reaction, cloning in a plasmid, RNA transcription, or the like, to provide sufficient material for subsequent analysis.

Oligonucleotide tags are employed for two different purposes in certain embodiments of the invention: (1) oligonucleotide tags are employed to implement solid phase cloning, as described in Brenner, U.S. Pat. No. 5,604,097; and International patent application PCT/US96/09513, wherein large numbers of polynucleotides, e.g., several thousand to several hundred thousand, are sorted from a mixture into clonal subpopulations of identical polynucleotides on one or more solid phase supports for analysis; and (2) they are employed to deliver (or accept) labels to identify polynucleotides, such as encoded adaptors, that number in the range of a few tens to a few thousand, e.g., as disclosed in Albrecht et al., International patent application PCT/US97/09472. For the former use, large numbers, or repertoires, of tags are typically required, and therefore synthesis of individual oligonucleotide tags is difficult. In these embodiments, combinatorial synthesis of the tags is preferred. On the other hand, where extremely large repertoires of tags are not required-such as for delivering labels to a plurality of kinds or subpopulations of polynucleotides in the range of 2 to a few tens, e.g., encoded adaptors, oligonucleotide tags of a minimally cross-hybridizing set may be separately synthesized, as well as synthesized combinatorially.

Sets containing several hundred to several thousands, or even several tens of thousands, of oligonucleotides may be synthesized directly by a variety of parallel synthesis approaches, e.g., as disclosed in Frank et al., U.S. Pat. No. 4,689,405; Frank et al., Nucleic Acids Research 11:4365-4377 (1983); Matson et al., Anal. Biochem. 224:110-116 (1995); Fodor et al., International application PCT/US93/04145; Pease et al., Proc. Natl. Acad. Sci. 91:5022-5026 (1994); Southern et al., J. Biotechnology 35:217-227 (1994), Brennan, International application PCT/US94/05896; Lashkari et al., Proc. Natl. Acad. Sci. 92:7912-7915 (1995); or the like.

Preferably, tag complements in mixtures, whether synthesized combinatorially or individually, are selected to have similar duplex or triplex stabilities to one another so that perfectly matched hybrids have similar or substantially identical melting temperatures. This permits mismatched tag complements to be more readily distinguished from perfectly matched tag complements in the hybridization steps, e.g., by washing under stringent conditions. For combinatorially synthesized tag complements, minimally cross-hybridizing sets may be constructed from subunits that make approximately equivalent contributions to duplex stability as every other subunit in the set. Guidance for carrying out such selections is provided by published techniques for selecting optimal PCR primers and calculating duplex stabilities, e.g., Rychlik et al., (1989) Nucleic Acids Research 17:8543-8551 and 18:6409-6412 (1990); Breslauer et al. (1986), Proc. Natl. Acad. Sci. 83:3746-3750; Wetmur (1991), Crit. Rev. Biochem. Mol. Biol. 26:227-259; and the like. A minimally cross-hybridizing set of oligonucleotides can be screened by additional criteria, such as GC-content, distribution of mismatches, theoretical melting temperature, and the like, to form a subset which is also a minimally cross-hybridizing set.

The oligonucleotide tags of the invention and their complements are conveniently synthesized on an automated DNA synthesizer, e.g., an Applied Biosystems, Inc. (Foster City, Calif.) Model 392 or 394 DNA/RNA Synthesizer, using standard chemistries, such as phosphoramidite chemistry, e.g., disclosed in the following references: Beaucage and Iyer (1992), Tetrahedron 48:2223-2311; Molko et al, U.S. Pat. No. 4,980,460; Koster et al., U.S. Pat. No. 4,725,677; Caruthers et al., U.S. Pat. Nos. 4,415,732; 4,458,066; and 4,973,679; and the like.

Oligonucleotide tags for sorting may range in length from 12 to 60 nucleotides or basepairs. Preferably, oligonucleotide tags range in length from 18 to 40 nucleotides or basepairs. More preferably, oligonucleotide tags range in length from 25 to 40 nucleotides or basepairs. In terms of preferred and more preferred numbers of subunits, these ranges may be expressed as follows:

Numbers of Subunits in Tags in Preferred Embodiments

| Monomers in | Nueleotides in Oligonucleotide Tag | | |
|---|---|---|---|
| Subunit | (12-60) | (18-40) | (25-40) |
| 3 | 4-20 subunits | 6-13 subunits | 8-13 subunits |
| 4 | 3-15 subunits | 4-10 subunits | 6-10 subunits |
| 5 | 2-12 subunits | 3-8 subunits | 5-8 subunits |
| 6 | 2-10 subunits | 3-6 subunits | 4-6 subunits |

Most preferably, oligonucleotide tags for sorting are single stranded and specific hybridization occurs via Watson-Crick pairing with a tag complement.

Preferably, repertoires of single stranded oligonucleotide tags for sorting contain at least 100 members; more preferably, repertoires of such tags contain at least 1000 members; and most preferably, repertoires of such tags contain at least 10,000 members.

Preferably, the length of single stranded tag complements for delivering labels is between 8 and 20. More preferably, the length is between 9 and 15.

An exemplary tag library for sorting is shown below (SEQ ID NO:1).

```
Left Primer Bsp 120I
5′-AGAATTCGGGCCTTAATTAA ↓
5′-AGAATTCGGGCCTTAATTAA-[⁴(A, G, T)₈]-GGGCCC-
      TCTTAAGCCCGGAATTAATT-[⁴(T, C, A)₈]-CCCGGG-
            ↑ ↑
        Eco RI Pac I
                                      Bbs I BamHI
                                    -GCATAAGTCTTCXXX . . . XXXGGATCCGAGTGAT-3′
                                    -CGTATTCAGAAGXXX . . . XXXCCTAGGCTCACTA
                                                                XXXXXCCTAGGCTCACTA-5′
                                                                                          Right Primer
```

### Formula I

The flanking regions of the oligonucleotide tag may be engineered to contain restriction sites, as exemplified above, for convenient insertion into and excision from cloning vectors. Optionally, the right or left primers may be synthesized with a biotin attached (using conventional reagents, e.g., available from Clontech Laboratories, Palo Alto, Calif.) to facilitate purification after amplification and/or cleavage. Preferably, for making tag-fragment conjugates, the above library is inserted into a conventional cloning vector, such a pUC19, or the like. Optionally, the vector containing the tag library may contain a “stuffer” region, “XXX . . . XXX,” which facilitates isolation of fragments fully digested with, for example, Bam HI and Bbs I.

An important aspect of the invention is the sorting and attachment of populations of DNA sequences, e.g., from a cDNA reference library, to microparticles or to separate regions on a solid phase support such that each microparticle or region has substantially only one kind of sequence attached; that is, such that the DNA sequences are present in clonal subpopulations. This objective is accomplished by insuring that substantially all different DNA sequences have different tags attached. This condition, in turn, is brought about by taking only a sample of the full ensemble of tag-DNA sequence conjugates for analysis. It is acceptable that identical DNA sequences have different tags, as it merely results in the same DNA sequence being operated on or analyzed twice. Sampling can be carried out either overtly-for example, by taking a small volume from a larger mixture-after the tags have been attached to the DNA sequences; it can be carried out inherently as a secondary effect of the techniques used to process the DNA sequences and tags; or sampling can be carried out both overtly and as an inherent part of processing steps.

If a sample of n tag-DNA sequence conjugates are randomly drawn from a reaction mixture-as could be effected by taking a sample volume, the probability of drawing conjugates having the same tag is described by the Poisson distribution, P(r)=e^{-λ}(λ)r/r, where r is the number of conjugates having the same tag and λ=np, where p is the probability of a given tag being selected. If n=10⁶ and p=1/(1.6⁷ H 10⁷) (for example, if eight 4-base words described in Brenner et al. were employed as tags), then λ=0.0149 and P(2)=1.13H10⁴. Thus, a sample of one million molecules gives rise to an expected number of doubles well within the preferred range. Such a sample is readily obtained by serial dilutions of a mixture containing tag-fragment conjugates.

As used herein, the term “substantially all” in reference to attaching tags to molecules, especially polynucleotides, is meant to reflect the statistical nature of the sampling procedure employed to obtain a population of tag-molecule conjugates essentially free of doubles. Preferably, at least ninety-five percent of the DNA sequences have unique tags attached.

Preferably, DNA sequences are conjugated to oligonucleotide tags by inserting the sequences into a conventional cloning vector carrying a tag library. For example, cDNAs may be constructed having a Bsp 120 I site at their 5′ ends and after digestion with Bsp 120 I and another enzyme such as Sau 3A or Dpn II may be directionally inserted into a pUC19 carrying the tags of Formula I to form a tag-fragment library, which includes every possible tag-fragment pairing. A sample is taken from this library for amplification and sorting. Sampling may be accomplished by serial dilutions of the library, or by simply picking plasmid-containing bacterial hosts from colonies. After amplification, the tag-fragment conjugates may be excised from the plasmid.

After the oligonucleotide tags are prepared for specific hybridization, e.g., by rendering them single stranded as described above, the polynucleotides are mixed with microparticles containing the complementary sequences of the tags under conditions that favor the formation of perfectly matched duplexes between the tags and their complements. There is extensive guidance in the literature for creating these conditions. Exemplary references providing such guidance include Wetmur (1991), Critical Reviews in Biochemistry and Molecular Biology 26:227-259; Sambrook et al., Molecular Cloning: A Laboratory Manual, 2nd Edition (Cold Spring Harbor Laboratory, New York, 1989); and the like. Preferably, the hybridization conditions are sufficiently stringent so that only perfectly matched sequences form stable duplexes. Under such conditions, the polynucleotides specifically hybridized through their tags may be ligated to the complementary sequences attached to the microparticles. Finally, the microparticles are washed to remove polynucleotides with unligated and/or mismatched tags.

The specificity of hybridization of tags to their complements may be increased by taking a sufficiently small sample so that both a high percentage of tags in the sample are unique and the nearest neighbors of substantially all the tags in a sample differ by at least two words. This latter condition may be met by taking a sample that contains a number of tag-polynucleotide conjugates that is about 0.1 percent or less of the size of the repertoire being employed. For example, if tags are constructed with eight words a repertoire of 88, or about 1.6⁷ H 10⁷, tags and tag complements are produced. In a library of tag-DNA sequence conjugates as described above, a 0.1 percent sample means that about 16,700 different tags are present. If this sample were loaded directly onto a repertoire-equivalent of microparticles, or in this example a sample of 1.6⁷ H 10⁷ microparticles, then only a sparse subset of the sampled microparticles would be loaded. Preferably, loaded microparticles may be separated from unloaded microparticles by a FACS instrument using conventional protocols after DNA sequences have been fluorescently labeled and denatured. After loading and FACS sorting, the label may be cleaved prior to use or other analysis of the attached DNA sequences.

The following provides a more detailed explanation of how the fragments isolated in accordance with the invention are isolated and labeled using conventional techniques. A large number of light-generating labels are available for labeling fragments, including fluorescent, colorimetric, chemiluminescent, and electroluminescent labels. Generally, such labels produce an optical signal which may comprise an absorption frequency, an emission frequency, an intensity, a signal lifetime, or a combination of such characteristics. Preferably, fluorescent labels are employed, either by direct incorporation of fluorescently labeled nucleoside triphosphates or by indirect application by incorporation of a capture moiety, such as biotinylated nucleoside triphosphates or an oligonucleotide tag, followed by complexing with a moiety capable of generating a fluorescent signal, such as a streptavidin-fluorescent dye conjugate or a fluorescently labeled tag complement. Preferably, the optical signal detected from a fluorescent label is an intensity at one or more characteristic emission frequencies. Selection of fluorescent dyes and means for attaching or incorporating them into DNA strands is well known, e.g., DeRisi et al. (supra), Matthews et al. (1988) Anal. Biochem. 169:1-25; Haugland, Handbook of Fluorescent Probes and Research Chemicals (Molecular Probes₅ Inc., Eugene, 1992); Keller and Manak, DNA Probes, 2nd Edition (Stockton Press, New York, 1993); and Eckstein, editor, Oligonucleotides and Analogues: A Practical Approach (IRL Press, Oxford, 1991); Wetmur (1991), Critical Reviews in Biochemistry and Molecular Biology 26:227-259; Ju et al. (1995), Proc. Natl. Acad. Sci., 92:4347-4351 and Ju et al. (1996), Nature Medicine 2:246-249; and the like.

Preferably, light-generating labels are selected so that their respective optical signals can be related to the quantity of labeled DNA strands present and so that the optical signals generated by different light-generating labels can be compared. Measurement of the emission intensities of fluorescent labels is the preferred means of meeting this design objective. For a given selection of fluorescent dyes, relating their emission intensities to the respective quantities of labeled DNA strands requires consideration of several factors, including fluorescent emission maxima of the different dyes, quantum yields, emission bandwidths, absorption maxima, absorption bandwidths, nature of excitation light source(s), and the like. Guidance for making fluorescent intensity measurements and for relating them to quantities of analytes is available in the literature relating to chemical and molecular analysis, e.g., Guilbault, editor, Practical Fluorescence, Second Edition (Marcel Dekker, New York, 1990); Pesce et al., editors, Fluorescence Spectroscopy (Marcel Dekker, New York, 1971); White et al., Fluorescence Analysis: A Practical Approach (Marcel Dekker, New York, 1970); and the like. As used herein, the term “relative optical signal” means a ratio of signals from different light-generating labels that can be related to a ratio of differently labeled DNA strands of identical, or substantially identical, sequence that form duplexes with a complementary reference DNA strand. Preferably, a relative optical signal is a ratio of fluorescence intensities of two or more different fluorescent dyes.

Competitive hybridization between the labeled DNA strands derived from different pools of individuals is carried out by applying equal quantities of the labeled DNA strands from each such source to the microparticles loaded with the reference DNA population in a conventional hybridization reaction. The particular amounts of labeled DNA strands added to the competitive hybridization reaction vary widely depending on the embodiment of the invention. Factors influencing the selection of such amounts include the quantity of microparticles used, the type of microparticles used, the loadings of reference strands on the microparticles, reaction volume, the complexity of the populations of labeled DNA strands, and the like. Hybridization is competitive in that differently labeled DNA strands with identical, or substantially identical, sequences compete to hybridize to the same complementary reference DNA strands. The competitive hybridization conditions are selected so that the proportion of labeled DNA strands forming duplexes with complementary reference DNA strands reflects, and preferably is directly proportional to, the amount of that DNA strand in its population in comparison with the amount of the competing DNA strands of identical sequence in their respective populations. Thus, if a first and second differently labeled DNA strands with identical sequence are competing for hybridization with a complementary reference strand such that the first labeled DNA strand is at a concentration of 1 ng/l and the second labeled DNA strand is at a concentration of 2 ng/l, then at equilibrium it is expected that one third of the duplexes formed with the reference DNA would include first labeled DNA strands and two thirds of the duplexes would include second labeled DNA strands. Guidance for selecting hybridization conditions is provided in many references, including Keller and Manak, (supra); Wetmur, (supra); Hames et al., editors, Nucleic Acid Hybridization: A Practical Approach (IL Press, Oxford, 1985); and the like.

Microparticles containing fluorescently labeled DNA strands are conveniently classified and sorted by a commercially available FACS instrument, e.g., Van Dilla et al., Flow Cytometry: Instrumentation and Data Analysis (Academic Press, New York, 1985). For fluorescently labeled DNA strands competitively hybridized to a reference strand, preferably the FACS instrument has multiple fluorescent channel capabilities. Preferably, upon excitation with one or more high intensity light sources, such as a laser, a mercury arc lamp, or the like, each microparticle will generate fluorescent signals, usually fluorescence intensities, related to the quantity of labeled DNA strands from each cell or tissue types carried by the microparticle.

Fragments carried by microparticles may be identified after sorting, e.g., by FACS, using conventional DNA sequencing protocols. Suitable templates for such sequencing may be generated in several different ways starting from the sorted microparticles carrying fragments of interest. For example, as illustrated in FIGS. 6A and 6B, the reference DNA attached to an isolated microparticle may be used to generate labeled extension products by cycle sequencing, e.g., as taught by Brenner, International application PCT/US95/12678. In this embodiment, primer binding site (600) is engineered into the reference DNA (602) distal to tag complement (606), as shown in FIG. 6A. After isolating a microparticle, e.g., by sorting into separate microtiter well, or the like, the differentially expressed strands are melted off, primer (604) is added, and a conventional Sanger sequencing reaction is carried out so that labeled extension products are formed. These products are then separated by electrophoresis, or like techniques, for sequence determination. In a similar embodiment, sequencing templates may be produced without sorting individual microparticles. Primer binding sites (600) and (620) may be used to generate templates by PCR using primers (604) and (622). The resulting amplicons containing the templates are then cloned into a conventional sequencing vector, such as M13. After transfection, hosts are plated and individual clones are selected for sequencing.

In another embodiment, illustrated in FIG. 6B, primer binding site (612) may be engineered into the competitively hybridized strands (610). This site need not have a complementary strand in the reference DNA (602). After sorting, competitively hybridized strands (610) are melted off of reference DNA (602) and amplified, e.g., by PCR, using primers (614) and (616), which may be labeled and/or derivatized with biotin for easier manipulation. The melted and amplified strands are then cloned into a conventional sequencing vector, such as M13, which is used to transfect a host which, in turn, is plated. Individual colonies are picked for sequencing.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference.

### EXAMPLES

### Example 1

### Isolation of Taq I-Polymorphic Fragments From a Sau 3A-Digested pUC19 in the Presence and Absence of Phage Lambda DNA

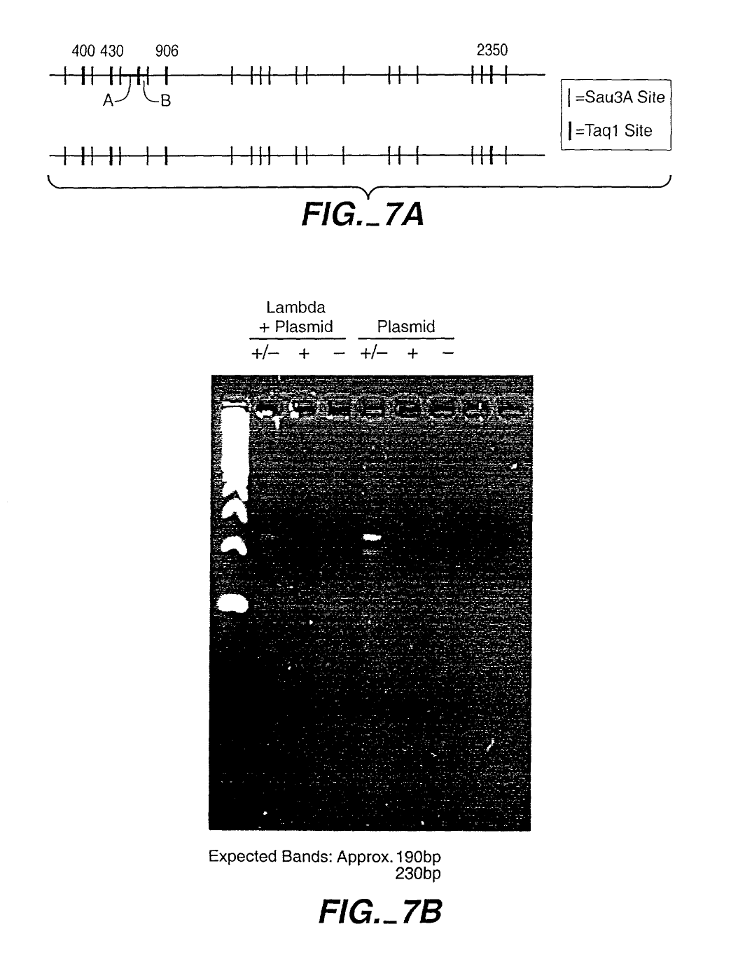

In this example, a conventional pUC19 plasmid was modified to create two additional Sau 3A sites between the Taq I sites located at base positions 430 and 906 of the plasmid (FIG. 7A). This newly created plasmid (p0T2S) was then modified further with the addition of a Taq I site between the two new Sau 3A sites, to create the plasmid p1T2S. Thus, the two plasmids are polymorphic at the new Taq 1 site. The two plasmids were digested separately with Sau 3A.

Single stranded portions of Sau 3A fragments containing Taq I sites (Taq+ fragments) were generated with the protocol outlined in FIG. 8A using the adaptors and primers whose sequences are listed below. The Sau 3A digested p1T2S plasmid (800) was filled in with dGTP and then an excess of Q adaptors was added (802) in a conventional ligation reaction to form product (804), which was then digested with Taq 1 (806) to give three possible products (808), (810), and (812). To this mixture, an excess of M adaptors were added (814) in a conventional ligation reaction to form the three possible products (816), (818), and (820). Preferably, M adaptors have the following two structural features: (i) 5′ extensions as shown below to prevent digestion by exonuclease III, and (ii) a protruding strand of three nucleotides at the end which is ligated to San 3A fragments digested by Taq I, thereby leaving a gap between one strand of the adaptor and the fragment it is ligated to. This latter feature ensures that fragments with two M adaptors (i.e., Taq I-Taq I fragments (820)) will not be amplified by PCR. After ligating M adaptors, the mixture was treated with exonuclease III (822) to render fragments (816) and (818) single stranded. M and Q primers were then added to the reaction mixture and PCR was carried out (824) to form product (826) which was then digested with Sau 3A (828) to remove the Q adaptor. The resulting fragment (830) was then treated (832) with T7 gene 6 5′-exonuclease to produce the single stranded fragments (834).

Single stranded portions of Sau 3A fragments lacking Taq I sites (Taq fragments) were generated from the plasmid p0T2S with the protocol outlined in FIG. 8B using the adaptors and primers whose sequences are listed below. The Sau 3A digested p0T2S was filled in with dGTP and then an excess of N adaptors were added (852) in a conventional ligation reaction to form product (854), which was then digested with Taq I (856) to give three possible products (858), (860), and (862). Preferably, the 5′ ends of the N adaptors are rendered resistant to exonuclease digestion by providing phosphorothioate linkages or other protecting modifications. The reaction mixture was then treated with T7 gene 6 exonuclease to render all fragments single stranded, except those (858) having two N adaptors attached. After treatment with exonuclease I (866) to eliminate single stranded fragments, N primers were added to the reaction mixture and PCR was carried out (868) to enrich the mixture for fragment (858). The resulting fragments were then treated (860) with exonuclease III to produce the single stranded fragments (862).

As illustrated in FIG. 8C, using the protocols given below, fragments (834) and (862) from the above reactions were annealed (870) and the 3′ strands of the resulting duplexes (872) were extended with T4 DNA polymerase (874) to form fragments (876) having primer binding sites for M and N primers. M and N primers were added to the reaction mixture and fragments (876) were copied by PCR. The PCR amplicons from the reaction were separated by gel electrophoresis and two fragments (190 basepairs and a 230 basepairs) were identified (lane +/! under “Plasmid”) which correspond to the portions A and B of the Sau 3A fragment illustrated in FIG. 7A.

The above experiment was repeated with the following alteration: an amount of phage lambda DNA equimolar to the pUC19 plasmid DNA was added to the initial Sau 3A digestion reaction. After carrying out the reactions outlined in FIGS. 8A-8C, the resulting fragments were separated by gel electro-phoresis and bands were identified (lane +/! under “Lambda+Plasmid”) which corresponded to the portions A and B of the Sau 3A fragment illustrated in FIG. 7A.

The sequences for the Q, N and M adaptors are as follows:

```
Q adaptor:
5′!ggtacagacatggaggtgcagactaaaa
          ccaugucuguaccuccacgucugauuuucuap;
N adaptor:
5′!tagtactcgtaatcagtgcttcaatgta
            atcatgagcattagtcacgaagttacatctap; and
M adaptor:
              5′!gtctccacgtcttattctgt
      tgtgagaagcagaggtgcagaataagacaagcp.
```

The sequences of the primers used for PCR include:

Q.top (=Q primer): 5′-ggtacagacatggaggtgcagactaaaa; N.top (=N primer): 5′-tagtactcgtaatcagtgcttcaatgta; and Mn.amp (=M primer): 5′-Cacactcttcgtctccacgtcttat.

### Example 2

### Isolation of Tai I-Polymorphic Fragments From a BstYI-Digested Human Genomic DNA

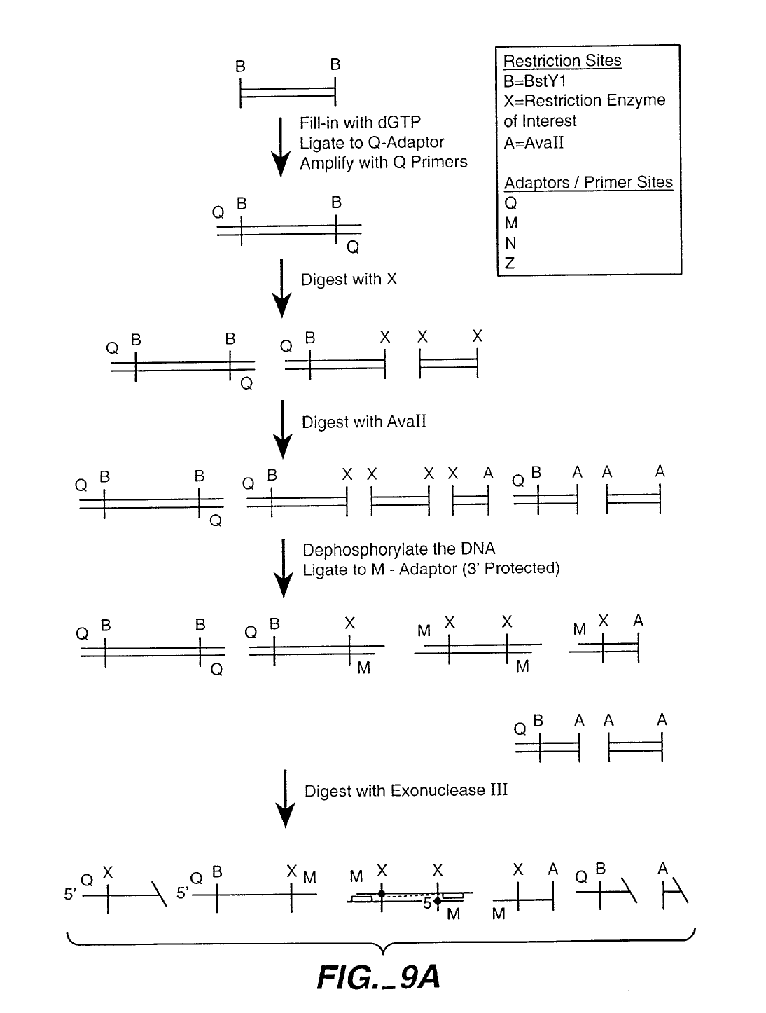

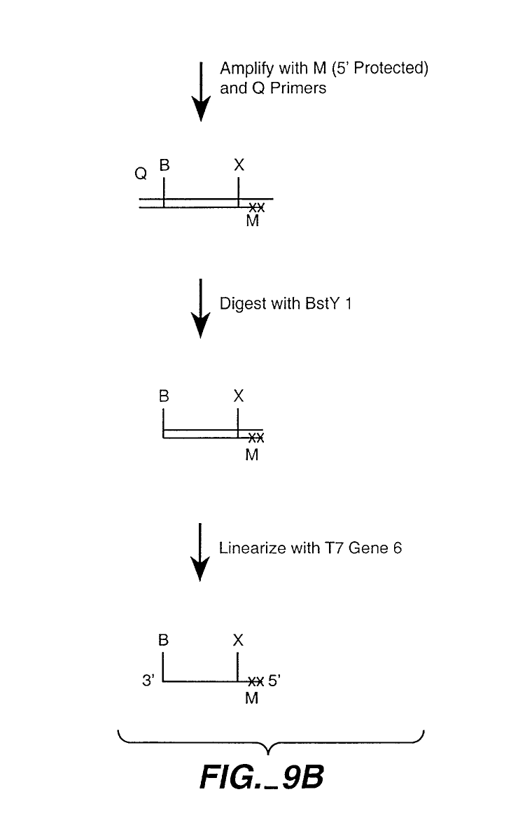

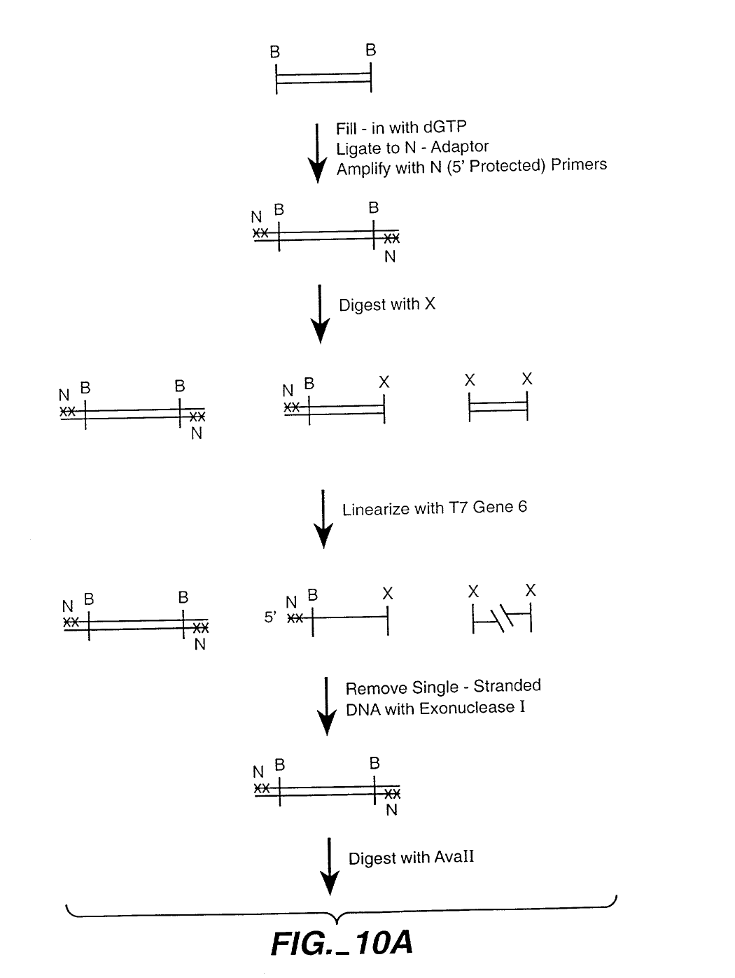

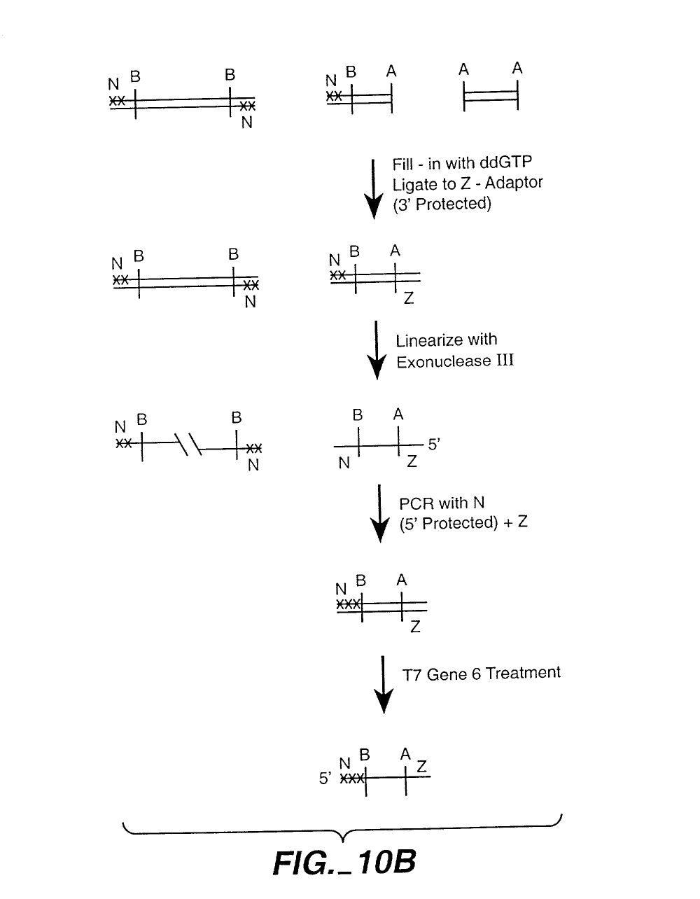

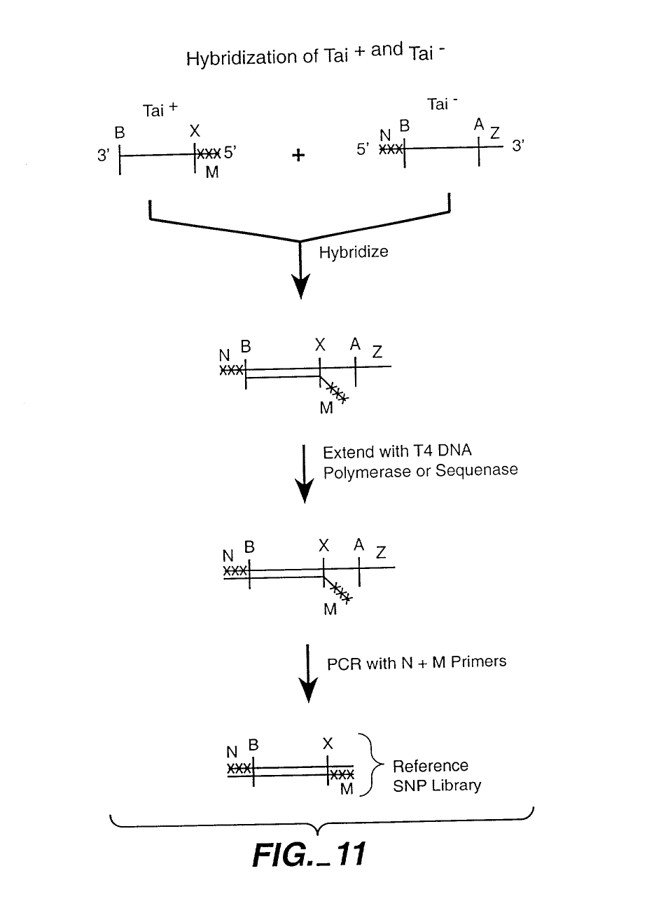

In this example, a first sample of genomic DNA was obtained and pooled from white blood cells isolated from a population of five diabetic patients. Separately, a second sample of genomic DNA was obtained and pooled from white blood cells isolated from a population of five normal individuals. Genomic DNA from white cells was isolated from whole blood by the protocol given below. Equal amounts of DNA from the first and second samples were combined in order to isolate Bst YI fragments (“Bst YI reference fragments”) capable of containing Tai I restriction site polymorphisms. Two aliquots were removed from the combined DNA samples and were separately digested to completion with Bst YI using the manufacturer's recommended protocol. Bst YI fragments containing Tai I sites (“Tai⁺ fragments”) were isolated from one aliquot by the protocol outlined in FIGS. 9A and 9B, and Bst YI fragments lacking Tai I sites (“Tai⁻ fragments”) were isolated from the other aliquot by the protocol outlined in FIGS. 10A and 10B. A reference population of polymorphic fragments was then generated by combining the Tai⁺ and Tai⁻ fragments as described in FIG. 11, after which the reference population may be cloned into a tag-containing vector, such as pNCV, as described below to form a library of tagged reference fragments. After transfection and expansion in an appropriate cloning vector, a sample is taken for further amplification and loading onto microparticles. Population-specific probe are then constructed as described above for identification of polymorphic sequences associated with either population.

The following is a more detailed description of the methods used to isolate Tail polymorphic fragments. First, genomic DNA is isolated and purified from Buffy-coat Preparations as follows: If the starting whole blood is 5-10 ml than you can expect approximately 10 H 10⁶! 60 H 10⁶ enriched leukocytes. Dilute the buffy-coat preparation at least 1/100 in phosphate-buffered saline (PBS) to determine the number of cells. There will probably be a small amount of erythrocytes in the preparation. Do not use more than 2 H 10⁷ cells per 100/G genomic tip column (Qiagen genomic DNA kit, cat #13343). Bring the buffy-coat preparation to 5 ml with cold PBS for up to 2 H 10⁷ cells in a 50 ml conical tube. Add 1 volume ice cold buffer lysis buffer (C1-Qiagen kit) and 3 volumes ice cold distilled water. Mix tube gently by inversion several times until the suspension is translucent. Incubate on ice for 10 minutes. Centrifuge the lysed enriched leukocytes at 4° C. for 15 minutes at 1300× g. Discard the supernatant. Repeat wash using 1 ml C1 and 3 ml distilled water until the pellet is white (indicating that residual hemoglobin has been removed). At this point, the washed pellet can be stored at −20° C. without loss in yield. If continuing the protocol, resuspend the pellet in 5 ml buffer G2 (Qiagen genomic DNA kit) and vortex the nuclei at high speed for 10-30 seconds. Add 95 μl of Qiagen protease and incubate at 50° C. for 30-60 minutes. The lysate should become clear at this stage. If not, extend the incubation time or pellet the undissolved material at 5000× g 10 minutes 4 C. The sample should be loaded onto the Qiagen genomic tip promptly.

To purify the DNA, equilibrate a Qiagen genomic-tip 100/G with 4 ml of Buffer QBT (Qiagen kit) using gravity flow. Vortex the genomic DNA sample for 10 seconds at maximum speed and apply it to the equilibrated column. Wash the Qiagen genomic tip twice with 7.5 ml Qiagen Buffer QC. Elute DNA with 5 ml of Qiagen Buffer QF. Add 3.5 ml room-temperature isopropanol and mix the tubes 10-20 times to precipitate the DNA. Dissolve the DNA pellet in water (100-200 μl) overnight on a shaker, or at 55 C for a few hours. After DNA is dissolved, dilute it 1:50 and measure the optical density (OD) at 260/280. The ratio for blood cells may be low due to residual hemoglobin. The yield should be approximately 50-200 ug.

Single-stranded Tai⁺ Bst Y1 fragments are prepared by filling-in with dGTP. Ethanol precipitated BstY1 digested mixed genomic DNA is filled in with dGTP, in order to prevent concatenation of fragments in the following ligation step. To fill-in with dGTP, mix: 2 μl 10× Klenow buffer (500 mM Tris. HCl pH 7.5, 100 mM MgCl₂, 10 mM DTT); 500 ng BstY1 digested (ethanol precipitated) genomic DNA; 0.4 μl 1.65 mM dGTP; 0.5 μl 5 U/μl Klenow (Exo-); and H2O to a final volume of 20 μl. Incubate at 37° C. for 30 minutes and inactivate at 75° C. for 10 minutes.

Q adaptors are ligated to both ends of the filled-in BstY1 fragments, thereby maintaining the BstY1 site. To ligate to Q adaptor, mix the following: 4 μl 5× LB1 (125 mM Tris. HCl pH 8.0, 22.5 mM DTT); 10 μl DNA; 1 μl 10 μM adaptor; 2 μl 2 mM ATP; 2.5 mM H₂O; and 0.5 μl 2000 U/μl T4 DNA ligase, in a final volume of 20 μl and incubate at 16° C. overnight.

In order to produce unmethylated DNA, so that methylation-sensitive restriction enzymes (e.g., Taq I) will cut to completion, the DNA is amplified using Q-top primer. Conditions for PCR are 55 C annealing temperature; 35 cycles, 30 second extension, 100 μl reaction; 0.8 μM (i.e., 0.4 μM each end) primer; 2.5 mM final concentration MgCl2; using 1 μl template (from 20 μl ligation reaction).

To purify the DNA obtained following amplification, extract with phenol/chloroform/isoamylalcohol and then with chloroform/isoamylalcohol. Precipitate with ethanol (80% ethanol wash) and resuspend in 10 μl H₂O.

The purified DNA is then digested with Tai. To digest with Tai, mix the following: 1 μg DNA; 10 μl 10× Buffer R⁺ (MBI; 100 mM Tris (pH 8.5)), 100 mM MgCl₂, 1 M KCl 1 mg/ml BSA); H₂O to 98 μl; and 2 μl Tai in a final volume of 100 μl, and incubate at 65° C. for 5 hours.

Following digestion with Tai, the DNA is purified by extracting with phenol/chloroform/isoamylalcohol, followed by extraction with chloroform/isoamylalcohol. The DNA is then precipitated with ethanol (80% ethanol wash) and resuspended in 10 μl H₂O.

Next, the purified DNA is digested with Ava II. To digest with Ava II, mix the following: 10 μl 10× NEB4 (500 mM KOAc, 200 mM TrisOAc, 100 mM MgOAc, 10 mM DTT); 10 μl DNA; 2 μl Ava II (50 U/μl); and 78 μl H₂O to a final volume of 100 μl and incubate at 37° C. for 5 hours.

Dephosphorylating the DNA is necessary to prevent the formation of concatomers. To dephosphorylate the DNA, mix the following: 100 μl DNA; and 1 μl SAP (shrimp alkaline phosphatase) (1 U/μl) to a final volume of 101 μl. Incubate at 37° C. for 30 minutes and inactivate at 65° C. for 20 minutes.

Prior to ligating to the M adaptor, the DNA is purified. To purify the DNA, extract with phenol/chloroform/isoamylalcohol, and then extract with chloroform/isoamylalcohol. The DNA is precipitated with ethanol (80% ethanol wash) and resuspended in 10 μl H₂O.

Ligation to the M adaptor permits BstY1 fragments to be amplified while maintaining the Tai site. The 3′-end of the M adaptor is protected from exonuclease III.

To ligate to the M adaptor, mix the following: 4 μl 10× LB3 (250 mM Tris, pH 7.5), 25 mM MgCl₂, 25 mM DTT); 10,ul DNA; 0.5 μl 10 μM M-tai adaptor; 2 μl 2 mM ATP; 3 μl H₂O; 0.5 μl T4 DNA ligase (2000 U/μl) to a final volume of 20 μl and incubate at 16° C. overnight.

Next, the DNA is linearized with exonuclease III to produce single-stranded DNA. To treat the DNA with Exonuclease III, mix the following: 20 μl DNA; 1 μl ExoIII (100 U/μl) to a final volume of 20 μl, and incubate at 37° C. for 2 hours; then inactivate at 75° C. for 10 minutes.

The DNA fragments obtained after treatment with exonuclease III are amplified using ssssMN.amp and Q-top primers. Negative controls use M primer alone and Q primer alone. To amplify the DNA, mix together the following: 39.75 μl H2O; 5 μl 10× Taq buffer; 1 μl 10 mM dNTP; 1 μl template; 1 μl each 10 μM primer; 2 μl 25 mM MgCl₂ (2.5 mM final); and 0.25 μl HS Taq to a final volume of 50 μl. Amplify using the following conditions: 15 minute preheating step at 95° C., followed by 35 cycles of 94° C. for 30 seconds, 50° C. for 30 seconds, and 72° C. for 1 minute. A final step at 72° C. for 5 minutes.

Following amplification, the DNA is purified by extracting first with phenol/chloroform/isoamylalcohol, and then with chloroform/isoamylalcohol. The DNA is precipitated with ethanol (8% ethanol wash) and resuspended in 10 μl H₂O.

To remove the Q adaptor, the DNA from above is digested with BstY1. To digest with Bst Y1, mix the following: 2 μl 10× Bst Y1 buffer (NEB; 100 mM Tris, pH 7.9, 100 mM MgCl₂, 10 mM DTT); 0.2 μl 10 mg/ml BSA; 10 μl DNA; 6.8 μl H₂O; and 1 μl BstY1 (20 U/μl) in a final volume of 20 μl and incubated at 60° C. for 2 hours.

After removal of the Q adaptor, the DNA is linearized with T7 gene 6. To treat the DNA with T7gene6, mix together the following: 20 μl DNA; 19 μl H₂O; and 1 μl T7 gene 6 in a final volume of 40 μl. Incubate at 23° C. for 60 minutes and inactivate at 80° C. for 20 minutes to form single-stranded DNA ready for hybridization.

To produce single-stranded DNA consisting of all the Bst Y1 fragments that lack Tai restriction sites, it is important that the Tai-digestion step goes to completion, as uncut sites will be identified falsely as polymorphisms. First, ethanol precipitated Bst Y1 digested mixed genomic DNA is filled in with dGTP, in order to prevent concatenation of fragments in the following ligation step. To fill-in with dGTP, mix the following: 2 μl 10× Klenow buffer (250 mMTris.HCl pH 7.5, 100 mM MgCl₂, 10 mM DTT); 500 ng BstY1-digested (ethanol precipitated) genomic DNA; 0.4 μl 1.65 mM dGTP; 0.5 μl 5 U/μl Klenow (Exo-); H₂O to 20 μl to a final volume of 20 μl. Incubate at 37° C. for 30 minutes and inactivate at 75° C. for 10 minutes.

N adaptors are ligated to both ends of the filled-in Bst Y1 fragments, thereby maintaining the Bst Y1 site. 5′ protected adaptors are used. To ligate to N adaptor, mix the following: 4 μl 5× LBI (125 mM Tris.HCl pH 8.0, 22.5 mM DTT); 10 μl DNA; 1 μl 10 μM adaptor (=ssssN adaptor);

2 μl 2 mM ATP; 2.5 mM H₂O; and 0.5 μl 2000 U/μl T4 DNA ligase in a final volume of 20 μl and incubate at 16° C. overnight.

To produce unmethylated DNA, so that methylation-sensitive restriction enzymes (e.g., Taq I) will cut to completion, the DNA obtained from the previous step is amplified using ssssN-top primer. The conditions for amplification are: 50° C. annealing temperature; 35 cycles, 30 second extension; 100 μl reaction containing 0.8 μM (i.e., 0.4 μM each end) primer, 2.5 mM final concentration MgCl₂ and template from the 20 μl ligation reaction.

To purify the DNA following amplification, extract with phenol/chloroform/isoamylalcohol, followed by extraction with chloroform/isoamylalcohol. Precipitate with ethanol (80% ethanol wash) and resuspend in 10 μl H₂O.

The DNA purified from above is then digested with Tai. To digest with Tai, mix the following: 1 μg DNA; 10 μl 10× Buffer R⁺ (MBI; 100 mM Tris (pH 8.5), 100 mM MgCl₂, 1M KCl, 1 mg/ml BSA); H₂O to 98 μl; and 2 μl Tail in a final volume of 100 μl and incubate at 65° C. for 5 hours.

To prevent linear amplification of digested fragments, the DNA is first linearized with T7 gene 6 and then treated with exonuclease I. To treat the DNA with T7 gene 6, mix together the following: 100 μl DNA; and 1 μl T7 gene 6 in a final volume of 101 μl total.

Incubate at 23° C. for 30 minutes and inactivate at 70° C. for 25 minutes.

To treat the DNA with exonuclease I, mix together the following: 101 μl DNA and 1 μl exonuclease I in a final volume of 102 μl, incubate at 37° C. for 30 minutes and inactivate at 70° C. for 25 minutes.

Purify the DNA by extracting first with phenol/chloroform/isoamylalcohol, and then with chloroform/isoamylalcohol. Precipitate with ethanol (80% ethanol wash) and resuspend in 10 μl H₂O.

The purified DNA obtained from above is then digested with Ava II. To digest with Ava II, mix the following: 10 μl NEB4 (500 mM KOAc, 200 mM TrisOAc, 100 mM MgOAc, 10 mM DTT); 10 μl DNA; 79 μl H₂O; and 1 μl Ava I in a final volume of 100 μl, incubate at 37° C. for 5 hours and inactivate at 65° C. for 20 minutes. Following digestion with Ava II, the DNA is purified by extracting first with phenol/chloroform/isoamylalcohol, followed by chloroform/isoamylalcohol, precipitating with ethanol (80% ethanol wash), and resuspending in 20 μl H₂O.

Purified DNA from above is filled in with dGTP by mixing the following: 2 μl Klenow buffer (250 mM Tris.HCl pH 7.5, 100 mM MgCl₂, 10 mM DTT); 10 μl DNA; 0.4 μl 1.65 mM dGTP; 0.5 μl 5 U/μl Klenow (Exo-); and 7.1 μl H₂O in a final volume of 20 μl, incubating at 37° C. for 30 minutes and inactivating at 70° C. for 20 minutes.

Following the fill-in reaction with dGTP, the Z-adaptor is ligated onto the DNA fragments by mixing: 4 μl 5× LB1 (250 mM Tris.HCl pH 8.0, 22.5 mM DTT); 10 μl DNA; 1 μl 5 μl adaptor (=ZavaW adaptor); 2 μl 2 mM ATP; 2.5 mM H₂O; and 0.5 μl 2000U/μl T4 DNA ligase in a final volume of 20 μl, and incubating at 16° C. overnight.

After ligation of the Z-adaptors, the DNA is linearized with exonuclease III by mixing: 20 μl DNA and 1 μl exonuclease (100 U/μl) in a final volume of 21 μl, incubating at 37° C. for 2 hours and inactivating at 75° C. for 10 minutes.

To amplify those fragments that lack Tai sites, mix together the following: 38.75 μl H₂O; 5 μl 10× Taq Pol buffer; 1 μl 10 mg/ml dNTP; 1 μl 10 μM ssssN.top; 1 μl 10 μM Z.top; 2 μl 25 mM MgCl₂; 1 μl DNA; and 0.25 μl HS Taq in a final volume of 50 μl.

The DNA is then amplified under the following conditions: pretreating for 15 minutes at 95° C.; followed by 35 cycles at 94° C. for 30 seconds, 50° C. for 30 seconds, and 72° C. for 1 minute. A final step for 5 minutes is done at 72° C. The ssssN-top primer alone as negative control. The resulting DNA is purified by extracting first with phenol/chloroform, then chloroform, precipitating with ethanol, and resuspending in 10 μl H₂O.

The final step in obtaining single-stranded Tai⁻ fragments is to linearize the DNA with T7 gene 6. This step produces full-length N-Z (Tai) fragments and is important for preventing mispriming from unrelated repetitive sequences. To treat the DNA with T7 gene 6, mix together: 8 μl 5× T7 gene 6 buffer (200 mM Tris.HCl, pH 7.5, 100 mM MgCl₂, 250 mM NaCl); 10 μl DNA; 21 μl H₂O; and 1 μl T7 gene 6 in a final volume of 40 μl. Incubate at 23° C. for 60 minutes and inactivate at 75° C. for 10 minutes.

The polymorphic Tai⁻ and Tai⁺ single stranded fragments are rescued by first hybridizing and then amplifying using N and M primers. Only those fragments containing N and M adaptors (i.e., polymorphic fragments) should be amplified. Single stranded DNA samples are hybridized together by mixing the following: 4 μl Tail DNA; 4 μl Tai⁻ DNA; 12 μl 1 H Bst Y1 buffer (NEB); final volume 20 μl. The mixture is then incubated at 94° C. for 5 minutes, then cooled quickly on ice. Two ul of 1 M NaCl is added to give a final concentration of 0.1 M NaCl. The mixture is then incubated at 65° C. overnight.

Two μl of the hybridized DNA is removed and added to 0.1 μl 10 mg/ml dNTP; 1 μl 10P buffer (400 mM Tris 7.5, 200 mM MgCl₂, 500 mM NaCl); 0.8 μl sequenase; 6.1 μl H₂O; final volume 10 μl. The mixture is incubated at 37° C. for 30 minutes and deactivated at 75° C. for 10 minutes.

To amplify the DNA, the following is mixed together: 19.875 μl H₂O; 2.5 μl Taq buffer; 0.5 μl 10 mg/ml dNTP; 0.5 μl 10 μM N.top primter; 0.5 μl 10 μM BN.amp primer; 1 μl template (extended); 0.125 μl HS Taq; in a final volume of 25 μl.

The DNA is amplified under the following conditions: a 15 minute preheating step at 95° C.; followed by 35 cycles of 30 seconds at 94° C., 30 seconds at 50° C. and one minute at 72° C.; followed by a final step of 5 minutes at 72° C.

Adaptors used in this example are:

| | |
|---|---|
| Q | ggtacagacatggaggtgcagactaaaa |
| adaptor: | ccaugucuguaccuccacgucugauuuucuaP |
| ssssN | tagtactcgtaatcagtgcttcaatgta |
| adaptor: | atcatgagcattagtcacgaagttacatctaP |
| M | gtctccacgtcttattctgttcgacg |
| adaptor: | tgtgagaagcagaggtgcagaataagacaagcP |
| ZavaW | tttagaagcagactgtaagaccgt |
| adaptor: | tgtgagaagaaatcttcgtctgacattctggcacca/tP |

```
Primers used for PCR in this example are:
          Q.top: ggtacagacatggaggtgcagactaaaa;
          SsssN.top: tagtactcgtaatcagtgcttcaatgta;
          ssssMn.amp: acactcttcgtctccacgtcttat;
          Mn.amp: acactcttcgtctccacgtcttat; and
          Z.top: tttagaagcagactgtaagaccgtga.
```

Note: the nucleotides written in bold are phosphorothioates, which provide protection against T7 gene6 exonuclease (this is why the primers and adaptors have the ssss -to denote the four 5′ phosphorothioate nucleotides).

### Example 3

### Construction of an Eight-Word Tag Library

An eight-word tag library with four-nucleotide words was constructed from two two-word libraries in vectors pLCV-2 and pUCSE-2. Prior to construction of the eight-word tag library, 64 two-word double stranded oligonucleotides were separately inserted into pUC19 vectors and propagated. These 64 oligonucleotides consisted of every possible two-word pair made up of four-nucleotide words selected from an eight-word minimally cross-hybridizing set described in Brenner, U.S. Pat. No. 5,604,097. After the identities of the inserts were confirmed by sequencing, the inserts were amplified by PCR and equal amounts of each amplicon were combined to form the inserts of the two-word libraries in vectors, pLCV-2 and pUCSE-2. These were then used as described below to form an eight-word tag library in pUCSE, after which the eight-word insert was transferred to vector pNCV3 which contains additional primer binding sites and restriction sites to facilitate tagging and sorting polynucleotide fragments.

pUC19 was digested to completion with Sap I and Eco RI using the manufacturers protocol and the large fragment isolated to give pUCSE. All restriction endonucleases, unless otherwise noted, were purchased from New England Biolabs (Beverly, Mass.). The small Sap I-Eco RI fragment was removed to eliminate the β-gal promoter sequence, which was found to skew the representation of some combinations of words in the final library. The following adaptor (SEQ ID NO: 13) was ligated to the isolated large fragment in a conventional ligation reaction to give plasmid pUCSE as a ligation product.

EcoRI Pst I Eco RV Hind III
↓ ↓ ↓ ↓
aattctagactgcagttgatatcttaagctt
gatctgacgtcaactatagaattcgaacga

A bacterial host was transformed by the ligation product using electroporation, after which the transformed bacteria were plated, a clone selected, and the insert of its plasmid sequenced for confirmation. pUCSE isolated from the clone was then digested with Eco RI and Hind III using the manufacturer's protocol and the large fragment was isolated. The following adaptor (SEQ ID NO:14) was ligated to the large fragment to give plasmid pUCSE-D1 which contained the first di-word (underlined).

```
                            BseRI
  EcoRI PstI BbsI Bsp120I HindIII
        ↓ ↓ ↓ ↓ ↓ ↓
aattctgcagaggagatgaagacgaaaagaaaggggcccatgctgca
gacgtctcctctacttctgcttttctttccccgggtacgacgttcga
                                                                                                                                    ↑
                                                                                                                                BbvI
```

### Formula I

Further plasmids, pUCSE-D2 through pUCSE-D64, containing di-words were separately constructed from pUCSE-D1 by digesting it with Pst I and Bsp120 I and separately ligating the following adaptors (SEQ ID NO:15) to the large fragment.

gaggagatgaagacga[word][word]g
acgtctcctctacttctgct[word][word]cccgg

### Formula II

The words of the top strand were selected from the following minimally cross-hybridizing set: gatt, tgat, taga, tttg, gtaa, agta, atgt, and aaag. After cloning and isolation, the inserts of the vectors were sequenced to confirm the identities of the di-words.

Plasmid cloning vector pLCV-D1 was created from plasmid vector pBC.SK- (Stratagene) as follows, using the following oligonucleotides:

S-723 (SEQ ID NO:16)
5′-CGA GAA AGA GGG ATA AGG CTC GAG CTT AAT TAA GAG TCG ACG
AAT TCG GGC CCG GAT CCT GAC TCT TTC TCC CT-3′
S-724 (SEQ ID NO:17)
5′-CTA GAG GGA GAA AGA GTC AGG ATC CGG GCC CGA ATT CGT CGA CTC
TTA ATT AAG CTC GAG CCT TAT CCC TCT TTC TCG GTA C-3′
S-785 (SEQ ID NO:18)
5′-TCG AGG CAT AAG TCT TCG AAT TCC ATC ACA CTG GGA AGA CAA CGT
AG-3′
S-786 (SEQ ID NO:19)
5′-GAT CCT ACG TTG TCT TCC CAG TGT GAT GGA ATT CGA AGA CTT ATG
CC-3′
S-960 (SEQ ID NO:20)
5′-TCG ATT AAT TAA CAA GCT TTG GGC CCT CGA GCA TAA GTC TTC TGC
AGA ATT CGG ATC CAT CGA TGG TCA TAG C-3′
S-961 (SEQ ID NO:21)
5′-TGT TTC CTG CCA CAC AAC ATA CGA GCC GGA AGC GGC CGC TCT AGA-3′
S-962 (SEQ ID NO:22)
5′-AGC GTC TAG AGC GGC CGC TTC CGG CTC GTA TGT TGT GTG GCA GGA
AAC AGC TAT GAC CAT C-3′
S-963 (SEQ ID NO:23)
5′-GAT GGA TCC GAA TTC TGC AGA AGA CTT ATG CTC GAG GGC CCA AAG
CTT GTT AAT TAA-3′
S-1105 (SEQ ID NO:24)
5′-TCGA GGG CCC GCA TAA GTC TTC-3′
S-1106 (SEQ ID NO:25)
5′-TCGA GAA GAC TTA TGC GGG CCC-3′

Oligonucleotides S-723 and S-724 were kinased, annealed together, and ligated to pBC.SK- which had been digested with KprI and XbaI and treated with calf intestinal alkaline phosphatase, to create plasmid pSW 143.1.

Oligonucleotides S-785 and S-786 were kinased, annealed together, and ligated to plasmid pSW143.1, which had been digested with XhoI and BamHI and treated with calf intestinal alkaline phosphatase, to create plasmid pSW164.02.

Oligonucleotides S-960, S-961, S-962, and S-963 were kinased and annealed together to form a duplex consisting of the four oligonucleotides. Plasmid pSW164.02 was digested with XhoI and SapI. The digested DNA was electrophoresed in an agarose gel, and the approximately 3045 bp product was purified from the appropriate gel slice. Plasmid pUC4K (from Pharmacia) was digested with PstI and electrophoresed in an agarose gel. The approximately 1240 bp product was purified from the appropriate gel slice. The two plasmid products (from pSW164.02 and pUC4K) were ligated together with the (S-960/961/962/963) duplex to create plasmid pLCVa.

DNA from Adenovirus5 (New England Biolabs) was digested with PacI and Bsp120I, treated with calf intestinal alkaline phosphatase, and electrophoresed in an agarose gel. The approximately 2853 bp product was purified from the appropriate gel slice. This fragment was ligated to plasmid pLCVa which had been digested with PacI and Bsp120I, to create plasmid pSW208.14.

Plasmid pSW208.14 was digested with XhoI, treated with calf intestinal alkaline phosphatase, and electrophoresed in an agarose gel. The approximately 5374 bp product was purified from the appropriate gel slice. This fragment was ligated to oligonucleotides S-1105 and S-1106 (which had been kinased and annealed together) to produce plasmid pLCVb, which was then digested with Eco RI and Hind III. The large fragment was isolated and ligated to the Formula I adaptor (SEQ ID NO:14) to give pLCV-D 1.

As above for pUCSE, further plasmids, pLCV-D2 through pLCV-D64, containing di-words were separately constructed from pLCV-D1 by digesting it with Pst I and Bsp120I, isolating the large fragment, and a ligating an adaptor of Formula II. After cloning and isolation, the inserts of the vectors were sequenced to confirm the identities of the di-words.

Each of the vectors pLCV-D1 through -D64 and pUCSE-D1 through -D64 was separately amplified by PCR. The components of the reaction mixture were as follows:

| | | |
|---|---|---|
| 10 | μl | template (about 1-5 ng) |
| 10 | μl | 10x Klentaq ™ buffer (Clontech Laboratories, Palo Alto, |
| | | Calif.) |
| 2.5 | μl | biotinylated DF primer at 100 pmoles/l |
| 2.5 | μl | biotinylated DR primer at 100 pmoles/l |
| 2.5 | μl | 10 mM deoxynucleoside triphosphates |
| 5 | μl | DMSO |
| 66.5 | μl | H₂O |
| 1 | μl | Advantage Klentaq ™ (Clontech Laboratories, Palo Alto, |
| | | Calif.) |

The temperature of the reactions was controlled as follows: 94° C. for 3 minutes; 25 cycles of 94° C. for 30 seconds, 60° C. for 30 seconds, and 72° C. for 10 seconds; followed by 72° C. for 3 minutes, then 4° C. The DF and DR primer binding sites were upstream and downstream portions of the vectors selected to give amplicons of 104 basepairs in length. After the reactions were completed, 5 μl of each PCR product were separated polyacrylamide gel electrophoresis (20% with 1× TBE) to confirm by visual inspection that the reaction yields were approximately the same for each PCR. After such confirmation, using conventional protocols, 10 μl of each PCR was extracted twice with phenol and once with chloroform, after which the DNA in the aqueous phase was precipitate with ethanol. After resuspension in 200 μl of 1× NEB buffer #2 (New England Biolabs, Beverly, Mass.), the DNA was cleaved with Bbv I and Eco RI by adding the enzymes in 50 μl of the manufacturer's recommended buffer. The digestion resulted in the production of three fragments: a biotinylated fragment of 38 basepairs, a di-word-containing fragment of 29 basepairs, and a biotinylated fragment of 37 basepairs. After completion of the reaction, the excess biotinylated primers were removed by adding 50 μl 50% Ultralink (streptavidin-Sepharose, Pierce Chemical Co., Rockford, Ill.) and vortexing the mixture at room temperature for 30 minutes. The Ultralink material was separated from the reaction mixture by centrifugation, after which approximately half of the mixture was separated by polyacrylamide gel electrophoresis (20% gel). The 29-basepair band was cut out of the gel and the 29-basepair fragment was eluted using the “crush and soak” method, e.g., Sambrook et al., Molecular Cloning, Second Edition (Cold Spring Harbor Laboratory, New York, 1989). This material was then ligated into either pLCV-D1 or pUCSE-D1 after the latter were digested with Bbs I and Eco RI and treated with calf intestinal alkaline phosphatase, using the manufacturer's recommend protocols.

pNCV3 was constructed by first assembling the following fragment (SEQ ID NO:26) from synthetic oligonucleotides:

```
                EcoRI
                    ↓
aattctgtaaaacgacggccagtcgccagggttttcccagtcacgacgtgaataaatag-
    gacattttgctgccggtcagcggtcccaaaagggtcagtgctgcacttatttatc-
                    PacI Bsp120I
                      ↓ ↓
      ttaattaaggaataggcctctcctcgagctcggtaccgggcccgcataagtcttc-
      aattaattccttatccggagaggagctcgagccatggcccgggcgtattcagaag-
                        ClaI EcoRV SapI BamHI
                          ↓ ↓ ↓ ↓
      atctatcgatgattgaagagcgatatcgctcttcaatcggatccatcc-
      tagatagctactaacttctcgctatagcgagaagttagcctaggtagg-
                                                      ↑
                                                    SapI
                                                                                                            HindIII
                                                                                                                  ↓
          tcaactaattaccacacaacatacgagccggaagcgggtcatagctgtttcctga
          agttgattaatggtgtgttgtatgctcggccttcgcccagtatcgacaaaggacttcga
```

After isolation, the fragment was cloned into Eco RI and Hind III-digested pLCV-Dl using conventional protocols.

The di-words of pLCV-2 were amplified either by PCR or plasmid expansion, the product was digested with Eco RI and BbvI after which the Eco RI-BbvI fragment was isolated as insert 1. Two-word library pUCSE-2 was digested with Eco RI, Bbs I, and Pst I, after which the large fragment was treated with calf intestinal alkaline phosphatase to give vector 1. Vector 1 and insert 1 were combined in a conventional ligation reaction to give three-word library, pUCSE-3. pUCSE-3 was digested with Eco RI, Bbs I, and Pst I, after which the large fragment was treated with calf intestinal alkaline phosphatase to give vector 2. Vector 2 and insert 1 were then combined in a conventional ligation reaction to give four-word library, pUCSE-4. The 4-mer words of pUCSE-4 were amplified either by PCR or plasmid expansion, the product was digested with Eco RI and BbvI after which the Eco RI-BbvI fragment was isolated as insert 2. pLCV-2 was digested with Eco RI, Bbs I, and Pst I, after which the large fragment was treated with calf intestinal alkaline phosphatase to give vector 3. Vector 3 and insert 2 were then combined in a conventional ligation reaction to give five-word library, pLCV-5. The 5-mer words of pLCV-5 were amplified either by PCR or plasmid expansion, the product was digested with Eco RI and BbvI after which the Eco RI-BbvI fragment was isolated as insert 3. pUCSE-4 was digested with Eco RI, Bbs I, and Pst I, after which the large fragment was treated with calf intestinal alkaline phosphatase to give vector 4. Vector 4 and insert 3 were then combined in a conventional ligation reaction to give eight-word library, pUCSE-8. The 8-mer words of pUCSE-8 were amplified either by PCR or plasmid expansion, the product was digested with Bse RI and Bsp120 I, after which the BseRI-Bsp120I fragment was isolated as insert 4. pNCV3 was digested with Bse RI, Bsp120 I, and Sac I, after which the large fragment was isolated and treated with calf intestinal alkaline phosphatase to give vector 5. Vector 5 was then combined with insert 4 in a conventional ligation reaction to give the eight-word library pNCV3-8.

What is claimed is:

1. A method of making a reference library comprising a mixture of heterogeneous nucleic acid fragments, comprising:
digesting pooled nucleic acids comprising first restriction sites with a first restriction endonuclease to produce a mixture of restriction fragments;
forming a first population of single stranded nucleic DNA fragments from a first subpopulation of said restriction fragments, wherein said first subpopulation of restriction fragments comprises a second restriction site which is different from said first restriction site;
forming a second population of single stranded DNA fragments from a second subpopulation of said restriction fragments wherein said second subpopulation of said restriction fragments do not contain said second restriction site, and wherein said first single stranded DNA fragments have complementary sequences to said second single stranded DNA fragments from said second subpopulation when said single stranded DNA fragments are derived from the same restriction fragment;
hybridizing the first and second populations of single stranded DNA fragments to form a first population of duplexes;
treating said first population of duplexes with a single strand dependent nuclease to digest mismatched duplexes in said first population.

2. The method of claim 1, further comprising:
reannealing intact single stranded DNA from said nuclease digestion to form a second population of duplexes; and
isolating said second population of duplexes to form a reference population of restriction fragments.

3. The method of claim 1, further comprising the step of amplifying the matched duplexes using PCR.

4. The method of claim 1, wherein said single strand dependent nuclease is selected from the group consisting of T7 endonuclease, S1 nuclease and mungbean nuclease.

5. The method of claim 1, wherein said single strand dependent nuclease is T7 endonuclease.

6. The method of claim 2, wherein said population of duplexes is isolated using biotin precipitation.

7. A method of making a reference library comprising a mixture of heterogeneous nucleic acid fragments, comprising:
digesting pooled nucleic acid comprising first restriction sites with a first restriction endonuclease to produce a mixture of first restriction fragments having first cleavage ends;
ligating an Exo III resistant linker to the first cleavage ends of said first restriction fragments to form a first ligation product;
digesting said first ligation product with a second restriction endonuclease to for m a mixture of second restriction fragments, some of which comprise a second cleavage end;
ligating an Exo III susceptible linker to said second cleavage ends of said second restriction fragments to form a second ligation product population which includes said first ligation product,
wherein said Exo III susceptible linker comprises a first member of a binding pair;
digesting said second ligation product population with Exo III to form a third ligation product population comprising (i) single stranded DNA comprising end sequences corresponding to said Exo III resistant and Exo III susceptible linkers and (ii) double stranded DNA comprising end sequences corresponding to said Exo III resistant linkers;
denaturing said third ligation product population and hybridizing the mixture so obtained to form a reannealed third ligation product population; and
contacting said annealed third ligation product population with a second member of said binding pair to enrich for duplexes which form a reference population of restriction fragments.

8. The method of claim 7, further comprising contacting said reannealed third ligation product with a single strand dependent nuclease.

9. The method of claim 7, further comprising contacting said reannealed third ligation product population with exonuclease I.

10. The method of claim 7, wherein said Exo III susceptible linker further comprises biotin.
